(19) United States
(12) Patent Application Publication
(10) Pub. No.: US 2025/0374741 A1
(43) Pub. Date: Dec. 4, 2025

(54) PHOTOELECTRIC CONVERSION ELEMENT, IMAGING ELEMENT, OPTICAL SENSOR, AND COMPOUND

(71) Applicant: FUJIFILM CORPORATION, Tokyo (JP)

(72) Inventors: Yuko Suzuki, Kanagawa (JP); Yosuke Yamamoto, Kanagawa (JP); Hiroki Sugiura, Kanagawa (JP)

(21) Appl. No.: 19/298,433

(22) Filed: Aug. 13, 2025

Related U.S. Application Data
(63) Continuation of application No. PCT/JP2024/008511, filed on Mar. 6, 2024.

(30) Foreign Application Priority Data
Mar. 8, 2023 (JP) 2023-035591

(51) Int. Cl. H10K 30/30; H10K 30/85; H10K 30/86; H10K 85/60

(52) U.S. Cl. CPC H10K 30/353; H10K 30/85; H10K 30/86; H10K 85/615; H10K 85/636; H10K 85/653; H10K 85/654; H10K 85/655; H10K 85/6572; H10K 85/6574; H10K 85/6576

(57) ABSTRACT

The present invention provides a photoelectric conversion element which has excellent response speed in a case of receiving blue and green light and has low electric field strength dependence of the response speed. In addition, the present invention provides an imaging element, an optical sensor, and a compound, which are related to the photoelectric conversion element. The photoelectric conversion element of the present invention includes, in the following order, a conductive film, a photoelectric conversion film, and a transparent conductive film, in which the photoelectric conversion film contains a compound represented by Formula (1).

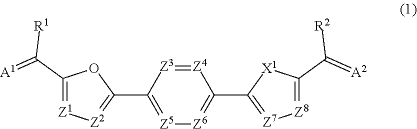

### CROSS-REFERENCE TO RELATED APPLICATIONS

This application is a Continuation of PCT International Application No. PCT/JP2024/008511 filed on Mar. 6, 2024, which claims priority under 35 U.S.C. § 119(a) to Japanese Patent Application No. 2023-035591 filed on Mar. 8, 2023. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element (for example, an imaging element) having a photoelectric conversion film has progressed.

For example, JP2019-508376A discloses an acceptor-donor-acceptor (ADA) type coloring agent which can be applied as a p-type semiconductor or an n-type semiconductor.

### SUMMARY OF THE INVENTION

In recent years, further improvements are also required for various characteristics required for a photoelectric conversion element used in an imaging element and an optical sensor, along with demands for improving performance of the imaging element, the optical sensor, and the like.

For example, there is a higher demand for the photoelectric conversion element to have excellent response speed in a case of receiving blue and green light (particularly, light having a wavelength of 460 nm) and to have low electric field strength dependence of the response speed.

Here, the above-described blue and green light refers to light in a wavelength range of 400 to 560 nm, and “low electric field strength dependence of the response speed” refers to a small change in response speed in a case where a voltage applied to the photoelectric conversion element is changed.

As a result of studying the photoelectric conversion element containing the compound, disclosed in JP2019-508376A, the present inventors have found that there is room for further improvement in response speed and electric field strength dependence of the response speed in a case of receiving the above-described blue and green light.

An object of the present invention is to provide a photoelectric conversion element which has excellent response speed in a case of receiving blue and green light and has low electric field strength dependence of the response speed.

Another object of the present invention is to provide an imaging element, an optical sensor, and a compound.

The present inventors have completed the present invention as a result of intensive studies to solve the above-described problems. That is, the present inventors have found that the above-described objects can be achieved by the following configuration.

[1]A photoelectric conversion element comprising, in the following order:
a conductive film;
a photoelectric conversion film; and
a transparent conductive film,
in which the photoelectric conversion film contains a compound represented by Formula (1) described later.

[2] The photoelectric conversion element according to [1],
in which at least one of Z¹, . . . , or Z⁸ is represented by —CR^{Z2}═,
provided that R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃, * represents a bonding position,
R^{Si1}'s each independently represent an aliphatic hydrocarbon group which may have a substituent, or an aromatic ring group which may have a substituent, and
the aliphatic hydrocarbon group and the acyl group, represented by R^{Z2}, and the aliphatic hydrocarbon group represented by R^{Si1} may have an ether oxygen atom.

[3] The photoelectric conversion element according to [2],
in which R^{Z2} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from a substituent group S described later, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃,
provided that the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have at least one of a halogen atom or an ether oxygen atom.

[4] The photoelectric conversion element according to [2] or [3],
in which R^{Z2} represents a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from a substituent group T described later, an acyl group having 2 or 3 carbon atoms, an aromatic ring group which may have a substituent selected from the substituent group T, and an aliphatic heterocyclic group which may have a substituent selected from the substituent group T.

[5] The photoelectric conversion element according to any one of [1] to [4],
in which the group represented by Formula (A-1) described later is a group represented by Formula (C-1) described later or a group represented by Formula (C-2) described later.

[6] The photoelectric conversion element according to [5],
in which the group represented by Formula (A-1) is the group represented by Formula (C-2).

[7] The photoelectric conversion element according to any one of [1] to [6],
in which X¹ is an oxygen atom.

[8] The photoelectric conversion element according to any one of [1] to [7],
in which the photoelectric conversion film further contains an n-type organic semiconductor, and
the photoelectric conversion film has a bulk heterojunction structure formed in a state in which the compound represented by Formula (1) and the n-type organic semiconductor are mixed with each other.

[9] The photoelectric conversion element according to [8],
in which the n-type organic semiconductor includes fullerenes selected from the group consisting of a fullerene and derivatives of the fullerene.

[10] The photoelectric conversion element according to any one of [1] to [9],
in which the photoelectric conversion film further contains a coloring agent.

[11] The photoelectric conversion element according to any one of [1] to [10],
in which the photoelectric conversion film further contains a p-type organic semiconductor.

[12] The photoelectric conversion element according to any one of [1] to [11], further comprising:
one or more interlayers between the conductive film and the transparent conductive film, in addition to the photoelectric conversion film.

[13] An imaging element comprising:
the photoelectric conversion element according to any one of [1] to [12].

[14] An optical sensor comprising:
the photoelectric conversion element according to any one of [1] to [12].

[15]A compound represented by Formula (1) described later.

[16] The compound according to [15],
in which at least one of Z¹, . . . , or Z⁸ is represented by —CR^{Z2}═,
provided that R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃, * represents a bonding position,
R^{Si1}'s each independently represent an aliphatic hydrocarbon group which may have a substituent, or an aromatic ring group which may have a substituent, and
the aliphatic hydrocarbon group and the acyl group, represented by R^{Z2}, and the aliphatic hydrocarbon group represented by R^{Si1} may have an ether oxygen atom.

[17] The compound according to [16],
in which R^{Z2} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from a substituent group S described later, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃,
provided that the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have at least one of a halogen atom or an ether oxygen atom.

[18] The compound according to [16] or [17],
in which R^{Z2} represents a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from a substituent group T described later, an acyl group having 2 or 3 carbon atoms, an aromatic ring group which may have a substituent selected from the substituent group T, and an aliphatic heterocyclic group which may have a substituent selected from the substituent group T.

[19] The compound according to any one of [15] to [18],
in which the group represented by Formula (A-1) described later is a group represented by Formula (C-1) described later or a group represented by Formula (C-2) described later.

[20] The compound according to [19],
in which the group represented by Formula (A-1) is the group represented by Formula (C-2).

[21] The compound according to any one of [15] to [20],
in which X¹ is an oxygen atom.

According to the present invention, it is possible to provide a photoelectric conversion element which has excellent response speed in a case of receiving blue and green light and has low electric field strength dependence of the response speed.

In addition, according to the present invention, it is possible to provide an imaging element, an optical sensor, and a compound.

### BRIEF DESCRIPTION OF THE DRAWINGS

FIG. 1 is a schematic cross-sectional view showing an example of a configuration of a photoelectric conversion element.

FIG. 2 is a schematic cross-sectional view showing an example of a configuration of a photoelectric conversion element.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, numerical ranges represented by “to” include numerical values before and after “to” as lower limit values and upper limit values.

In the present specification, a hydrogen atom may be a light hydrogen atom (normal hydrogen atom) or a heavy hydrogen atom (for example, a deuterium atom or the like).

In the present specification, regarding a compound which may have a geometric isomer (cis-trans isomer), a general formula or a structural formula representing the compound may be described only in the form of either a cis isomer or a trans isomer for convenience. Even in such a case, unless otherwise specified, the form of the compound is not limited to either the cis isomer or the trans isomer, and the compound may be the cis isomer or the trans isomer.

A bonding direction of a divalent group (for example, —CO—O—) described in the present specification is not limited unless otherwise specified. For example, in a case where Y in a compound represented by a formula “X—Y—Z” is —CO—O—, the compound may be any of “X—O—CO—Z” or “X—CO—O—Z”.

A symbol “*” specified in a chemical formula represents a bonding position unless otherwise specified.

In the present specification, in a case of a plurality of substituents, linking groups, and the like (hereinafter, also referred to as “substituent and the like”) represented by a specific reference numeral, or in a case of simultaneously defining a plurality of the substituent and the like, it means that each of the substituent and the like may be the same as or different from each other. This also applies to a case of specifying the number of substituents and the like.

In the present specification, the “substituent” includes a group exemplified by a substituent W described later, unless otherwise specified.

### (Substituent W)

The substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group (a heteroaryl group or an aliphatic heterocyclic group), a cyano group, a nitro group, an alkoxy group, an aryloxy group, a silyl group, a silyloxy group, a heterocyclic oxy group, an acyloxy group, a carbamoyloxy group, an alkoxycarbonyloxy group, an aryloxycarbonyloxy group, a primary, secondary, or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a carboxy group, a phosphoric acid group, a sulfonic acid group, a hydroxy group, a thiol group, an acylamino group, a carbamoyl group, a ureido group, and a boronic acid group.

In addition, each of the above-described groups may further have a substituent (for example, one or more groups of each of the above-described groups), as possible. For example, an alkyl group which may have a substituent is also included as the form of the substituent W.

In addition, in a case where the substituent W has a carbon atom, the number of carbon atoms in the substituent W is, for example, 1 to 20.

The number of atoms other than a hydrogen atom in the substituent W is, for example, 1 to 30.

It is also preferable that a specific compound described later does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, the aliphatic hydrocarbon group may be linear, branched, or cyclic.

Examples of the above-described aliphatic hydrocarbon group include an alkyl group, an alkenyl group, and an alkynyl group.

In addition, in the present specification, unless otherwise specified, the number of carbon atoms in the alkyl group is preferably 1 to 20, more preferably 1 to 10, and still more preferably 1 to 6.

The alkyl group may be linear, branched, or cyclic.

Examples of the alkyl group include a methyl group, an ethyl group, an n-propyl group, an isopropyl group, an n-butyl group, a t-butyl group, an n-hexyl group, and a cyclopentyl group.

In addition, the alkyl group may be any of a cycloalkyl group, a bicycloalkyl group, or a tricycloalkyl group, and may have a ring structure thereof as a partial structure.

In the alkyl group which may have a substituent, examples of the substituent which may be included in the alkyl group include the groups exemplified as the substituent W. Among these, an aryl group (preferably having 6 to 18 carbon atoms and more preferably having 6 carbon atoms), a heteroaryl group (preferably having 5 to 18 carbon atoms and more preferably having 5 or 6 carbon atoms), or a halogen atom (preferably a fluorine atom or a chlorine atom) is preferable.

In the present specification, unless otherwise specified, an alkyl group moiety in the alkoxy group is preferably the above-described alkyl group. An alkyl group moiety in the alkylthio group is preferably the above-described alkyl group.

In the alkoxy group which may have a substituent, examples of the substituent which may be included in the alkoxy group include the same examples as the substituent in the alkyl group which may have a substituent. In the alkylthio group which may have a substituent, examples of the substituent which may be included in the alkylthio group include the same examples as the substituent in the alkyl group which may have a substituent.

In the present specification, the alkenyl group may be any of linear, branched, or cyclic, unless otherwise specified. The number of carbon atoms in the above-described alkenyl group is preferably 2 to 20. In the alkenyl group which may have a substituent, examples of the substituent which may be included in the alkenyl group include the same examples as the substituent in the alkyl group which may have a substituent.

In the present specification, the alkynyl group may be any of linear, branched, or cyclic, unless otherwise specified. The number of carbon atoms in the above-described alkynyl group is preferably 2 to 20. In the alkynyl group which may have a substituent, examples of the substituent which may be included in the alkynyl group include the same examples as the substituent in the alkyl group which may have a substituent.

In the present specification, an aromatic ring or an aromatic ring constituting the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) fused aromatic ring structures, as a ring structure.

The number of ring member atoms in the above-described aromatic ring is preferably 4 to 15.

The above-described aromatic ring may be an aromatic hydrocarbon ring or an aromatic heterocyclic ring.

In a case where the above-described aromatic ring is an aromatic heterocyclic ring, the number of heteroatoms included as ring member atoms is, for example, 1 to 10. Examples of the above-described heteroatom include a nitrogen atom, a sulfur atom, an oxygen atom, a selenium atom, a tellurium atom, a phosphorus atom, a silicon atom, and a boron atom.

Examples of the above-described aromatic hydrocarbon ring include a benzene ring, a naphthalene ring, an anthracene ring, and a phenanthrene ring.

Examples of the above-described aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a pyrroloimidazole ring (for example, a 5H-pyrrolo[1,2-a]imidazole ring and the like), an imidazooxazole ring (for example, an imidazo[2,1-b]oxazole ring and the like), a thienothiazole ring (for example, a thieno[2,3-d]thiazole ring and the like), a benzothiadiazole ring, a benzodithiophene ring (for example, benzo[1,2-b:4,5-b′]dithiophene ring and the like), a thienothiophene ring (for example, thieno[3,2-b]thiophene ring and the like), a thiazolothiazole ring (for example, thiazolo[5,4-d]thiazole ring and the like), a naphthodithiophene ring (for example, a naphtho[2,3-b:6,7-b′]dithiophene ring, a naphtho[2,1-b:6,5-b′]dithiophene ring, a naphtho[1,2-b:5,6-b′]dithiophene ring, a 1,8-dithiadicyclopenta[b,g]naphthalene ring, and the like), a benzothienobenzothiophene ring, a dithieno[3,2-b:2′,3′-d]thiophene ring, and a 3,4,7,8-tetrathiadicyclopenta[a,e]pentalene ring.

In the aromatic ring which may have a substituent, examples of the type of the substituent which may be included in the aromatic ring include the groups exemplified as the substituent W. In a case where the above-described aromatic ring has a substituent, the number of substituents may be 1 or more (for example, 1 to 4 or the like).

In the present specification, examples of the “aromatic ring group” include a group obtained by removing one or more hydrogen atoms (for example, 1 to 5 or the like) from the above-described aromatic ring.

In the present specification, examples of the “aryl group” include a group obtained by removing one hydrogen atom from a ring corresponding to the aromatic hydrocarbon ring among the above-described aromatic rings.

In the present specification, examples of the “heteroaryl group” include a group obtained by removing one hydrogen atom from a ring corresponding to the aromatic heterocyclic ring among the above-described aromatic rings.

In the present specification, examples of the “arylene group” include a group obtained by removing two hydrogen atoms from a ring corresponding to the aromatic hydrocarbon ring among the above-described aromatic rings.

In the present specification, examples of the “heteroarylene group” include a group obtained by removing two hydrogen atoms from a ring corresponding to the aromatic heterocyclic ring among the above-described aromatic rings.

In the aromatic ring group which may have a substituent, the aryl group which may have a substituent, the heteroaryl group which may have a substituent, the arylene group which may have a substituent, and the heteroarylene group which may have a substituent, examples of the type of the substituent which may be included in these groups include the groups exemplified as the substituent W. In a case where these groups each of which may have a substituent have a substituent, the number of substituents may be 1 or more (for example, 1 to 4 or the like).

In the present specification, the number of ring members in the aliphatic heterocyclic group is preferably 5 to 20, more preferably 5 to 12, and still more preferably 6 to 8.

Examples of a heteroatom which is included in the above-described aliphatic heterocyclic group include a sulfur atom, an oxygen atom, a nitrogen atom, a selenium atom, a tellurium atom, a phosphorus atom, a silicon atom, and a boron atom; and a sulfur atom, an oxygen atom, or a nitrogen atom is preferable.

Examples of an aliphatic heterocyclic ring constituting the above-described aliphatic heterocyclic group include a pyrrolidine ring, an oxolane ring (tetrahydrofuran ring), a thiolane ring, a piperidine ring, a tetrahydropyran ring, a thiane ring (pentamethylene sulfide ring), a piperazine ring, a morpholine ring, a quinuclidine ring, an azetidine ring, an oxetane ring, an aziridine ring, a dioxane ring, and a γ-butyrolactone ring.

In the present specification, examples of the “aliphatic heterocyclic group” include a group obtained by removing one hydrogen atom from the above-described aliphatic heterocyclic group.

### [Photoelectric Conversion Element]

The photoelectric conversion element according to the embodiment of the present invention includes a conductive film, a photoelectric conversion film, and a transparent conductive film in this order, in which the photoelectric conversion film contains a compound represented by Formula (1) described later (hereinafter, referred to as “specific compound”).

The mechanism by which the photoelectric conversion element according to the embodiment of the present invention can achieve the above-described problems is not necessarily clear, but the present inventors assume as follows.

The mechanism by which the effect is obtained is not limited by the following supposition. That is, even in a case where the effect is obtained by a mechanism other than the following, it is included in the scope of the present invention.

The specific compound contained in the photoelectric conversion element according to the embodiment of the present invention is an A-D-A type coloring agent in which two acceptor sites (A) are bonded to a donor site (D). Since the above-described specific compound absorbs light to generate an exciton, a current can be taken out through charge separation and charge transport.

It is presumed that the specific compound according to the present invention improves element performance such as the response speed of the photoelectric conversion element by optimizing the structure of each of the donor site and the acceptor sites.

First, the donor site in the specific compound according to the present invention is characterized in that a furan ring and an aromatic 6-membered ring structure are bonded to each other. Due to steric hindrance between aromatic rings, a structure in which a 5-membered aromatic ring and a 6-membered aromatic ring are bonded to each other by a single bond usually has a twisted structure.

In a case where the donor site in the above-described A-D-A type coloring agent has such a twisted structure, an increase in rearrangement energy and the like occurs, which is disadvantageous in photoelectric conversion.

It is presumed that the specific compound according to the present invention reduces the above-described twist by bonding the furan ring and the aromatic 6-membered ring, and thus the performance such as the response speed and the electric field strength dependence of the response speed is improved. The reason why the twist between aromatic rings is small in a case where the furan ring is used is presumed to be that an oxygen atom of the furan ring has a small atomic radius and thus steric repulsion is small as compared with other atoms such as sulfur in a thiophene ring.

In addition, the structure of the acceptor site also has a significant effect on the element performance. For example, in the case of the A-D-A type coloring agent disclosed in JP2019-508376A, it is presumed that a dicyanomethylidene acceptor having a large dipole moment serves as a charge trapping agent, and the element performance (quantum efficiency, response speed, electric field strength dependence of response speed, and the like) is lowered.

On the other hand, it is presumed that the specific compound according to the present invention exhibits high element performance by suppressing charge trapping using a cyclic acceptor having a small dipole moment at the terminal.

Hereinafter, at least one of the effect that the quantum efficiency in a case where the photoelectric conversion element receives blue and green light (light in a wavelength range of 400 to 560 nm) is more excellent or the effect that the electric field strength dependence of the response speed is low is also referred to as the fact that the effect of the present invention is more excellent.

A configuration of the photoelectric conversion element according to the embodiment of the present invention will be described in detail below.

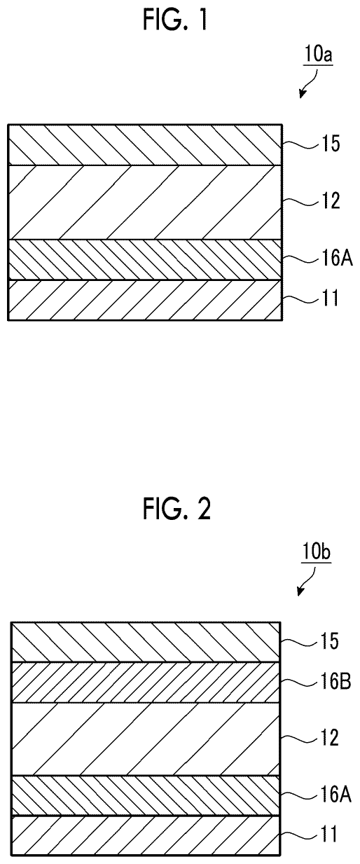

FIG. 1 shows a schematic cross-sectional view of one embodiment of the photoelectric conversion element according to the embodiment of the present invention.

A photoelectric conversion element 10a shown in FIG. 1 has a configuration in which a conductive film (hereinafter, also referred to as “lower electrode”) 11 functioning as a lower electrode, an electron blocking film 16A, a photoelectric conversion film 12 containing the specific compound, and a transparent conductive film (hereinafter, also referred to as “upper electrode”) 15 functioning as an upper electrode are laminated in this order.

FIG. 2 shows a configuration example of another photoelectric conversion element. A photoelectric conversion element 10b shown in FIG. 2 has a configuration in which the electron blocking film 16A, the photoelectric conversion film 12, a hole blocking film 16B, and the upper electrode 15 are laminated on the lower electrode 11 in this order. The lamination order of the electron blocking film 16A, the photoelectric conversion film 12, and the hole blocking film 16B in FIGS. 1 and 2 may be appropriately changed according to the application and the characteristics.

In the photoelectric conversion element 10a (or 10b), it is preferable that light is incident to the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of performance and power consumption, the applied voltage is more preferably 1×10⁻⁴ to 1×10⁷ V/cm and still more preferably 1×10⁻³ to 5×10⁶ V/cm.

Regarding the voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case of being incorporated in an imaging element, the voltage can be applied by the same method.

As described in detail below, the photoelectric conversion element 10a (or 10b) can be suitably applied to applications of an imaging element.

Hereinafter, the form of each layer constituting the photoelectric conversion element according to the embodiment of the present invention will be described in detail.

### [Photoelectric Conversion Film]

The photoelectric conversion element according to the embodiment of the present invention includes a photoelectric conversion film.

### Specific Compound

The photoelectric conversion film contains a compound represented by Formula (1) (specific compound).

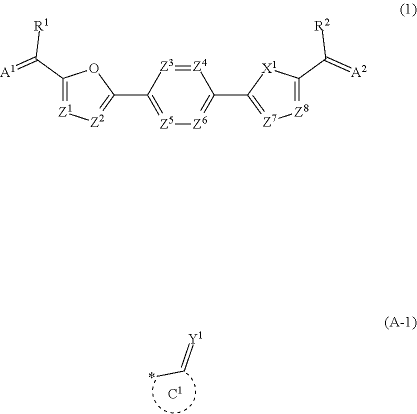

In Formula (1), X¹ represents an oxygen atom, a sulfur atom, or a selenium atom. As X¹, an oxygen atom or a sulfur atom is preferable, and an oxygen atom is more preferable.

In Formula (1), Z¹ to Z⁸ each independently represent —CR^{Z1}═ or —N═. R^{Z1} represents a hydrogen atom or a substituent.

In Formula (1), it is preferable that at least one of Z¹, . . . , or Z⁸ represents —CR^{Z1}═, it is more preferable that at least five thereof represent —CR^{Z1}═, and it is still more preferable that all of Z¹ to Z⁸ represent —CR^{Z1}═.

Among these, from the viewpoint that the effect of the present invention is more excellent, it is preferable that any one or two of Z¹ to Z⁸ (preferably any one or two of Z² to Z⁷) is —CR^{Z1}═ in which R^{Z1} is a substituent, and the rest of Z¹ to Z⁸ is —CR^{Z1}═ in which R^{Z1} is a hydrogen atom or a nitrogen atom.

In Formula (1), a plurality of R^{Z1}'s may be the same or different from each other.

Examples of the substituent represented by R^{Z1} include the groups exemplified as the substituent W, and a group represented by R^{Z2} described later is preferable. Among these, from the viewpoint that the effect of the present invention is more excellent, it is preferable that at least one of Z¹, . . . , or Z⁸ in Formula (1) is represented by —CR^{Z2}═

R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃. The above-described aliphatic hydrocarbon group and the described acyl group, represented by R^{Z2}, and the above-described aliphatic hydrocarbon group represented by R^{Si1} may have an ether oxygen atom. In a case where the above-described aliphatic hydrocarbon group has an ether oxygen atom, the aliphatic hydrocarbon group may have the ether oxygen atom between carbon atoms in the aliphatic hydrocarbon group, or may have the ether oxygen atom at a terminal of the aliphatic hydrocarbon group.

The substituent which may be included in each group represented by R^{Z2} is not particularly limited, and examples thereof include the groups exemplified as the substituent W. Among the substituent which may be included in each group represented by R^{Z2}, a substituent selected from the substituent group S described in detail later is preferable, and a substituent selected from the substituent group T described in detail later is more preferable. In addition, each group represented by R^{Z2} may have two or more substituents.

The number of carbon atoms in the above-described aliphatic hydrocarbon group is not particularly limited, and is preferably 1 to 20.

The definition of the aliphatic hydrocarbon group in the present specification is as described above, and is not particularly limited, but an alkyl group is preferable.

The number of carbon atoms in the linear aliphatic hydrocarbon group is not particularly limited, but is preferably 1 to 5, more preferably 1 to 3, and still more preferably 1 or 2.

The number of carbon atoms in the branched aliphatic hydrocarbon group is not particularly limited, but is preferably 3 to 10, more preferably 3 to 5, and still more preferably 3 or 4.

The number of carbon atoms in the cyclic aliphatic hydrocarbon group is not particularly limited, but is preferably 3 to 10, more preferably 3 to 8, and still more preferably 3 to 6.

As the substituent which may be included in the linear aliphatic hydrocarbon group and the branched aliphatic hydrocarbon group, a halogen atom or a group represented by *—Si(R^{Si1})₃ described later is preferable. In addition, as the substituent which may be included in the cyclic aliphatic hydrocarbon group, a substituent selected from the substituent group S is preferable, a substituent selected from the substituent group T is more preferable, and an alkyl group having 1 to 3 carbon atoms is still more preferable.

In a case where the aliphatic hydrocarbon group has a halogen atom, all hydrogen atoms in the aliphatic hydrocarbon group may be completely halogenated.

The number of carbon atoms in the above-described acyl group is not particularly limited, but is preferably 2 to 10, more preferably 2 to 5, and still more preferably 2 or 3.

The acyl group may be an aromatic acyl group or an aliphatic acyl group, and an aliphatic acyl group is preferable. Examples of the aliphatic acyl group include a group in which a carbon atom in the above-described aliphatic hydrocarbon group is substituted with a carbonyl carbon (C═O).

Among these, a linear aliphatic acyl group is preferable as the above-described acyl group.

As the substituent which may be included in the acyl group, a halogen atom is preferable.

In the present specification, the definition of the aromatic ring constituting the aromatic ring group is as described above, and examples of the aromatic ring group include an aryl group and a heteroaryl group.

The number of ring members in the aromatic ring group is not particularly limited, but is preferably 5 to 20 and more preferably 5 to 10.

The above-described aryl group is preferably a phenyl group which may have a substituent.

As the aromatic heterocyclic ring constituting the above-described heteroaryl group, thiophene which may have a substituent, furan which may have a substituent, or pyridine which may have a substituent is preferable.

As the substituent which may be included in the aromatic ring group, a substituent selected from the substituent group S is preferable, a substituent selected from the substituent group T is more preferable, and an alkyl group having 1 to 3 carbon atoms is still more preferable.

The definition of the aliphatic heterocyclic group in the present specification is as described above.

The number of ring members in the aliphatic heterocyclic group is not particularly limited, but is preferably 5 to 20, more preferably 5 to 12, and still more preferably 5 to 8.

As the aliphatic heterocyclic ring constituting the aliphatic heterocyclic group, pyrrolidine is preferable.

As the substituent which may be included in the aliphatic heterocyclic group, a substituent selected from the substituent group S is preferable, a substituent selected from the substituent group T is more preferable, and an alkyl group having 1 to 3 carbon atoms is still more preferable.

In the group represented by *—Si(R^{Si})₃, R^{Si1}'s each independently represent an aliphatic hydrocarbon group which may have a substituent or an aromatic ring group which may have a substituent.

The number of carbon atoms in the aliphatic hydrocarbon group represented by R^{Si1} is preferably 1 to 10 and more preferably 1 to 6.

The definition of the aliphatic hydrocarbon group in the present specification is as described above. Among these, an alkyl group is preferable as the aliphatic hydrocarbon group.

The number of ring members in the aromatic ring group represented by R^{Si1} is preferably 5 to 20 and more preferably 5 to 10.

Examples of the aromatic ring group include an aryl group and a heteroaryl group.

Examples of the substituent which may be included in the aliphatic hydrocarbon group or the aromatic ring group represented by R^{Si1} include the groups exemplified as the substituent W.

The group represented by *—Si(R^{Si1})₃ is more preferably a group represented by *—Si(R^{Si2})₃. R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S.

The above-described linear aliphatic hydrocarbon group having 1 to 3 carbon atoms, the above-described branched aliphatic hydrocarbon group having 3 or 4 carbon atoms, and the above-described cyclic aliphatic hydrocarbon group having 3 to 8 carbon atoms, represented by R^{Si2}, may have at least one of a halogen atom or an ether oxygen atom.

The above-described substituent group S is a group consisting of the following substituents.

Substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by *—Si(R^{Si2})₃

The above-described linear aliphatic hydrocarbon group having 1 to 3 carbon atoms, the above-described branched aliphatic hydrocarbon group having 3 or 4 carbon atoms, and the above-described cyclic aliphatic hydrocarbon group having 3 to 8 carbon atoms in the substituent group S may have at least one of a halogen atom or an ether oxygen atom.

The definition of the aliphatic hydrocarbon group in the present specification is as described above. Among these, an alkyl group is preferable as the aliphatic hydrocarbon group.

The above-described substituent group T is a group consisting of the following substituents.

Substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by *—Si(R^{Si3})₃

R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

The definition of the aliphatic hydrocarbon group in the present specification is as described above. Among these, an alkyl group is preferable as the aliphatic hydrocarbon group.

The above-described specific compound (compound represented by Formula (1)) is preferably a compound represented by any one of Formula (1-1) to Formula (1-6), more preferably a compound represented by any one of Formula (1-1) to Formula (1-5), still more preferably a compound represented by any one of Formula (1-1) to Formula (1-3), and particularly preferably a compound represented by Formula (1-1) or Formula (1-2).

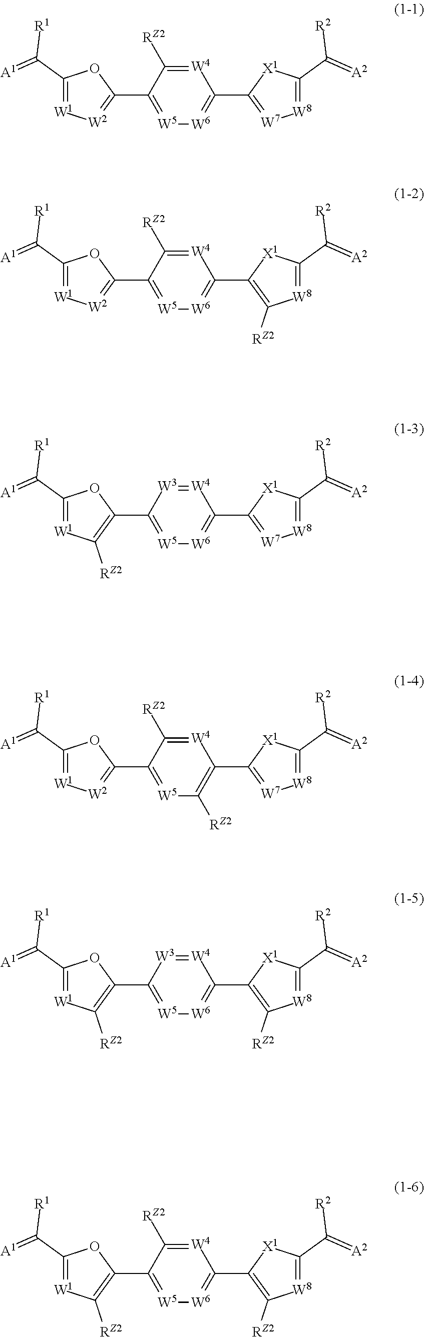

In Formulae (1-1) to (1-6), X¹, R¹, R², A¹, and A² have the same meanings as X¹, R¹, R², A¹, and A² in Formula (1).

R¹, R², A¹, and A² will be described in detail later.

W¹ to W⁸ each independently represent —CR^{Z1}═ or —N═. R^{Z1} has the same meaning as R^{Z1} in Formula (1). R^{Z1} is preferably a hydrogen atom, a fluorine atom, or a chlorine atom, and more preferably a hydrogen atom.

In Formulae (1-1) to (1-6), R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃, Specific aspects and suitable aspects of each group represented by R^{Z2} are as described above.

In Formula (1), R¹ and R² each independently represent a hydrogen atom or a substituent.

Examples of the substituent represented by R¹ and R² include the groups exemplified as the substituent W. Among these, from the viewpoint that the effect of the present invention is more excellent, R¹ and R² are each preferably a hydrogen atom.

In Formula (1), A¹ and A² each independently represent the above-described group represented by Formula (A-1).

In Formula (A-1), Y¹ represents a sulfur atom, an oxygen atom, ═NR^{Y1}, or ═CR^{Y2}R^{Y3}.

R^{Y1} represents a hydrogen atom or a substituent. R^{Y2} and R^{Y3} each independently represent a cyano group, —SO₂R^{Y4}, —COOR^{Y5}, or —COR^{Y6}.

From the viewpoint that the effect of the present invention is more excellent, Y¹ is an oxygen atom or a sulfur atom.

Examples of the substituent represented by R^{Y1} include the substituents exemplified as the substituent W.

In addition, R^{Y4} to R^{Y6} each independently represent an aliphatic hydrocarbon group which may have a substituent, an aromatic ring group which may have a substituent, or an aliphatic heterocyclic group which may have a substituent.

Examples of the substituent which may be included in the groups represented by R^{Y4} to R^{Y6} include the substituents exemplified as the substituent W.

The definition of the above-described aliphatic hydrocarbon group is as described above; and among these, an alkyl group is preferable, and a linear alkyl group is more preferable. The number of carbon atoms in the aliphatic hydrocarbon group is preferably 1 to 3.

The definition of the above-described aromatic ring group is as described above; and among these, an aryl group is preferable, and a phenyl group is more preferable.

The definition of the above-descried aliphatic heterocyclic group is as described above.

In Formula (A-1), C¹ represents a ring which contains two or more carbon atoms and may have a substituent.

The number of carbon atoms in the above-described ring is preferably 3 to 30, more preferably 3 to 20, and still more preferably 3 to 10. The above-described number of carbon atoms is a number including two carbon atoms specified in the formula.

The above-described ring may be aromatic or non-aromatic.

The above-described ring may be any of a monocyclic ring or a polycyclic ring, and is preferably a 5-membered ring, a 6-membered ring, or a fused ring including at least one of a 5-membered ring or a 6-membered ring. The number of rings forming the fused ring is preferably 1 to 4 and more preferably 1 to 3.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C¹, a carbon atom other than the carbon atom at a bonding position to which * is attached in Formula (A-1) and the carbon atom bonded to Y¹ may be replaced with a carbonyl carbon (>C═O) or a thiocarbonyl carbon (>C═S).

Examples of the substituent which may be included in the ring include the groups exemplified as the substituent W; and a halogen atom, an alkyl group, an aromatic ring group, or a silyl group is preferable, and a halogen atom or an alkyl group is more preferable.

The above-described alkyl group may be linear, branched, or cyclic, and is preferably linear.

The number of carbon atoms in the above-described alkyl group is preferably 1 to 10 and more preferably 1 to 3.

In Formula (A-1), as the above-described ring represented by C¹, a ring used as an acidic nucleus (for example, an acidic nucleus of a merocyanine coloring agent) is preferable; and examples thereof include the following nuclei.
(a) 1,3-dicarbonyl nucleus: for example, 1,3-indandione nucleus, 1,3-cyclohexanedione, 5,5-dimethyl-1,3-cyclohexanedione, 1,3-dioxane-4,6-dione, and the like
(b) pyrazolinone nucleus: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like
(c) isoxazolinone nucleus: for example, 3-phenyl-2-isoxazolin-5-one, 3-methyl-2-isoxazolin-5-one, and the like
(d) oxindole nucleus: for example, 1-alkyl-2,3-dihydro-2-oxindole and the like
(e) 2,4,6-trioxohexahydropyrimidine nucleus: for example, barbituric acid, 2-thibarbituric acid and derivatives thereof, and the like; examples of the above-described derivatives include a 1-alkyl form such as 1-methyl and 1-ethyl, a 1,3-dialkyl form such as 1,3-dimethyl, 1,3-diethyl, and 1,3-dibutyl, a 1,3-diaryl form such as 1,3-diphenyl, 1,3-di(p-chlorophenyl), 1,3-di(p-ethoxycarbonylphenyl), a 1-alkyl-1-aryl form such as 1-ethyl-3-phenyl, and a 1,3-diheteroaryl form such as 1,3-di(2-pyridyl).
(f) 2-thio-2,4-thiazolidinedione nucleus: for example, rhodanine and derivatives thereof, and the like; examples of the above-described derivatives include a 3-alkylrhodanine such as 3-methylrhodanine, 3-ethylrhodanine, and 3-allylrhodanine, a 3-arylrhodanine such as 3-phenylrhodanine, and a 3-heteroaryl rhodanine such as 3-(2-pyridyl)rhodanine.
(g) 2-thio-2,4-oxazolidinedione (2-thio-2,4-(3H,5H)-oxazoledione) nucleus: for example, 3-ethyl-2-thio-2,4-oxazolidinedione and the like
(h) thianaphthenone nucleus: for example, 3(2H)-thianaphthenone-1,1-dioxide and the like
(i) 2-thio-2,5-thiazolidinedione nucleus: for example, 3-ethyl-2-thio-2,5-thiazolidinedione and the like
(j) 2,4-thiazolidinedione nucleus: for example, 2,4-thiazolidinedione, 3-ethyl-2,4-thiazolidinedione, 3-phenyl-2,4-thiazolidinedione, and the like
(k) thiazolin-4-one nucleus: for example, 4-thiazolinone, 2-ethyl-4-thiazolinone, and the like
(l) 2,4-imidazolidinedione (hydantoin) nucleus: for example, 2,4-imidazolidinedione, 3-ethyl-2,4-imidazolidinedione, and the like
(m) 2-thio-2,4-imidazolidinedione (2-thiohydantoine) nucleus: for example, 2-thio-2,4-imidazolidinedione, 3-ethyl-2-thio-2,4-imidazolidinedione, and the like
(n) imidazolin-5-one nucleus: for example, 2-propylmercapto-2-imidazolin-5-one and the like
(o) 3,5-pyrazolidinedione nucleus: for example, 1,2-diphenyl-3,5-pyrazolidinedione, 1,2-dimethyl-3,5-pyrazolidinedione, and the like
(p) benzothiophen-3(2H)-one nucleus: for example, benzothiophen-3(2H)-one, oxobenzothiophen-3(2H)-one, dioxobenzothiophen-3(2H)-one, and the like
(q) indanone nucleus: for example, 1-indanone, 3-phenyl-1-indanone, 3-methyl-1-indanone, 3,3-diphenyl-1-indanone, 3,3-dimethyl-1-indanone, and the like
(r) benzofuran-3-(2H)-one nucleus: for example, benzofuran-3-(2H)-one and the like (s) 2,2-dihydrophenalene-1,3-dione nucleus and the like

From the viewpoint that the effect of the present invention is more excellent, the above-described group represented by Formula (A-1) is preferably a group represented by Formula (A-2).

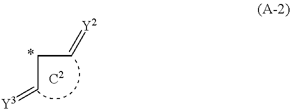

In Formula (A-2), * represents a bonding position.

Y² and Y³ each independently represent an oxygen atom, a sulfur atom, ═NR^{Y1}, or ═CR^{Y2}R^{Y3}.

R^{Y1} represents a hydrogen atom or a substituent. R^{Y2} and R^{Y3} each independently represent a cyano group, —SO₂R^{Y4}, —COOR^{Y5}, or —COR^{Y6}.

It is preferable that both Y² and Y³ represent an oxygen atom.

Examples of the substituent represented by R^{Y1} include the substituents exemplified as the substituent W.

In addition, R^{Y4} to R^{Y6} each independently represent an aliphatic hydrocarbon group which may have a substituent, an aromatic ring group which may have a substituent, or an aliphatic heterocyclic group which may have a substituent.

Examples of the substituent which may be included in the groups represented by R^{Y4} to R^{Y6} include the substituents exemplified as the substituent W.

The definition of the above-described aliphatic hydrocarbon group is as described above; and among these, an alkyl group is preferable, and a linear alkyl group is more preferable. The number of carbon atoms in the aliphatic hydrocarbon group is preferably 1 to 3.

The definition of the above-described aromatic ring group is as described above; and among these, an aryl group is preferable, and a phenyl group is more preferable.

In addition, in Formula (A-2), C² represents a ring which contains three or more carbon atoms and may have a substituent.

Three carbon atoms contained in C² are the three carbon atoms specified in Formula (A-2).

The number of carbon atoms in the above-described ring is preferably 3 to 30, more preferably 3 to 20, and still more preferably 3 to 10. The number of carbon atoms in the above-described ring is the number including the three carbon atoms specified in the formula. The above-described ring may be an aromatic ring or a non-aromatic ring.

The above-described ring may be any of a monocyclic ring or a polycyclic ring, and is preferably a 5-membered ring, a 6-membered ring, or a fused ring including at least one of a 5-membered ring or a 6-membered ring. In a case where the ring is a polycyclic ring, the number of rings included is preferably 2 to 6 and more preferably 2 or 3.

The above-described ring may have a heteroatom. Examples of the heteroatom include a nitrogen atom, a sulfur atom, an oxygen atom, a selenium atom, a tellurium atom, a phosphorus atom, a silicon atom, and a boron atom; and a sulfur atom, a nitrogen atom, or an oxygen atom is preferable.

The number of heteroatoms in the above-described ring is preferably 0 to 10 and more preferably 0 to 5.

Among the carbon atoms constituting the ring represented by C², a carbon atom other than the carbon atom at a bonding position to which * is attached in Formula (A-2) and the carbon atom bonded to Y² and Y³ may be replaced with a carbonyl carbon (>C═O) or a thiocarbonyl carbon (>C═S).

A suitable aspect of the substituent which may be included in the ring is the same as that of the substituent which may be included in the ring C¹ described above.

In addition, it is preferable that the above-described group represented by Formula (A-2) is a group represented by Formula (C-1) or a group represented by Formula (C-2).

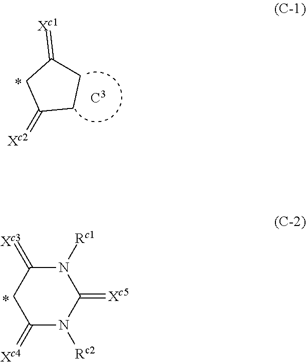

In Formula (C-1), X^{c1} and X^{c2} each independently represent an oxygen atom, a sulfur atom, ═NR^{X1}, or ═CR^{X2}R^{X3}.

R^{X1} represents a hydrogen atom or a substituent. R^{X2} and R^{X3} each independently represent a cyano group, —SO₂R^{X4}, —COOR^{X5}, or —COR^{X6}.

R^{X4}, R^{X5}, and R^{X6} each independently represent an aliphatic hydrocarbon group which may have a substituent, an aromatic ring group which may have a substituent, or an aliphatic heterocyclic group which may have a substituent.

It is preferable that at least one of X^{c1} or X^{c2} is an oxygen atom, and it is more preferable that both X^{c1} and X^{c2} are oxygen atoms.

In Formula (C-1), C³ represents an aromatic ring which may have a substituent. The number of carbon atoms in the above-described aromatic ring is preferably 4 to 30, more preferably 5 to 12, and still more preferably 6 to 8. The above-described number of carbon atoms is a number including two carbon atoms specified in the formula.

The above-described aromatic ring may be monocyclic or polycyclic.

In addition, the aromatic ring may be any of an aromatic hydrocarbon ring or an aromatic heterocyclic ring, and an aromatic hydrocarbon ring is preferable.

Examples of the aromatic ring represented by C³ include the rings exemplified in the description of the aromatic ring described above.

Among these, as the aromatic ring represented by C³, a benzene ring, a naphthalene ring, an anthracene ring, or a pyrene ring is preferable, and a benzene ring is more preferable.

Examples of the substituent which may be included in the aromatic ring include the groups exemplified as the substituent W.

In Formula (C-2), X^{c3} to X^{c5} represent an oxygen atom, a sulfur atom, ═NR^{X1}, or ═CR^{X2}R^{X3}.

The definitions of R^{X1} to R^{X3} are as described above.

It is preferable that both X^{c3} and X^{c4} are oxygen atoms, and it is more preferable that all of X^{c3}, X^{c4}, and X^{c5} are oxygen atoms.

In addition, R^{c1} and R^{c2} each independently represent a hydrogen atom or a substituent. Examples of the substituent represented by R^{c1} and R^{c2} include the groups exemplified as the substituent W, and among these, an alkyl group or a phenyl group is preferable, and an alkyl group is more preferable.

The above-described phenyl group may further have a substituent, and examples thereof include the groups exemplified as the substituent W.

A molecular weight of the specific compound is preferably 400 to 1,200, more preferably 400 to 1,000, and still more preferably 500 to 800.

In a case where the molecular weight is in the above-described range, it is presumed that sublimation temperature of the specific compound is low, and thus the quantum efficiency is excellent also in a case where the photoelectric conversion film is formed at a high speed.

From the viewpoint of stability in a case of using the specific compound as a p-type organic semiconductor and matching of energy levels between the specific compound and an n-type organic semiconductor, an ionization potential of the specific compound in a single film is preferably −6.0 to −5.0 eV.

A maximal absorption wavelength of the specific compound is preferably in a wavelength range of 400 to 600 nm, and more preferably in a wavelength range of 400 to 500 nm.

The above-described maximal absorption wavelength is a value measured in a solution state (solvent: chloroform) by adjusting the absorption spectrum of the specific compound to a concentration such that the light absorbance is 0.5 to 1.0. However, in a case where the specific compound is not soluble in chloroform, a value measured by using the specific compound in which the specific compound is vapor-deposited and formed into a film state is defined as the maximal absorption wavelength of the specific compound.

The specific compound is particularly useful as a material of a photoelectric conversion film used for an imaging element, an optical sensor, or a photoelectric cell. The specific compound usually functions as a coloring agent in the photoelectric conversion film. In addition, the specific compound can also be used as a coloring material, a liquid crystal material, an organic semiconductor material, a charge transport material, a pharmaceutical material, and a fluorescent diagnostic material.

Specific examples of the specific compound are shown below, but the present invention is not limited thereto.

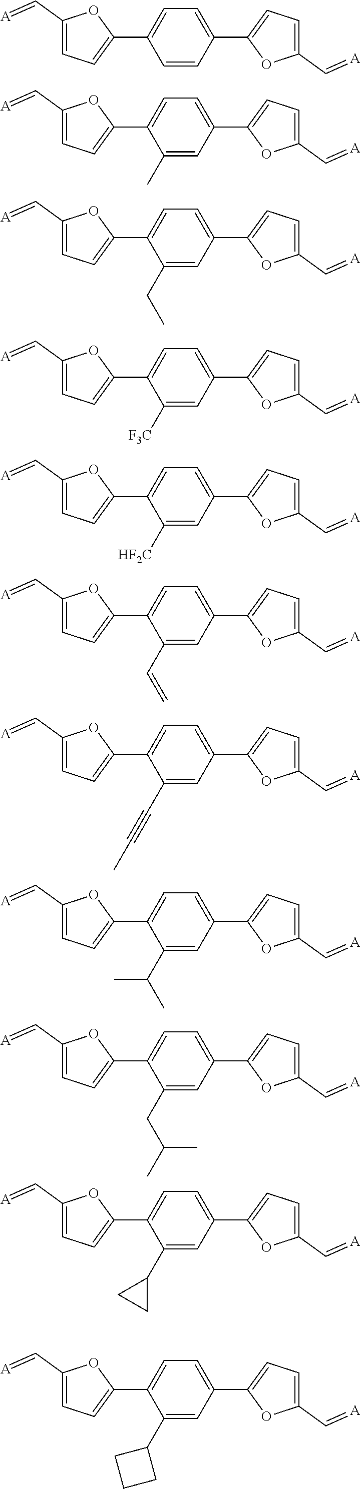

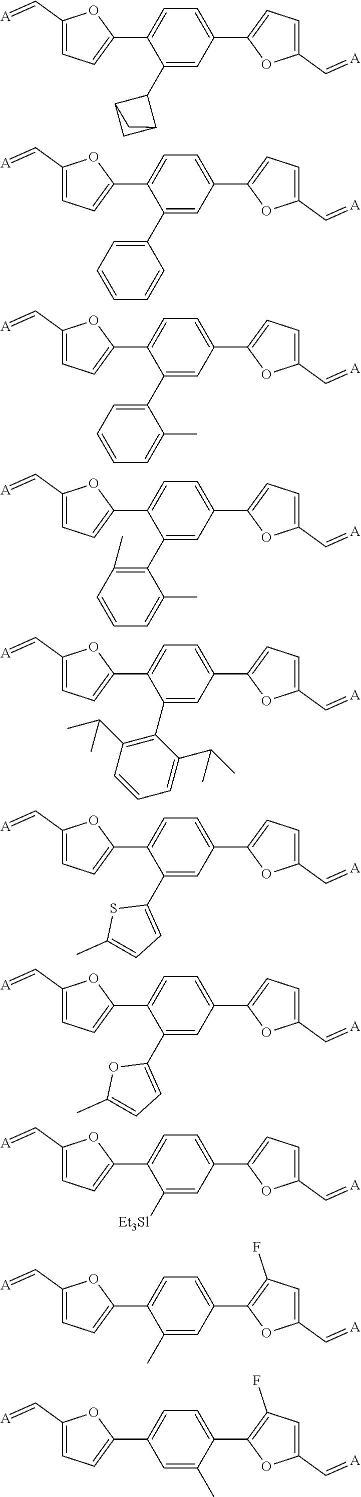

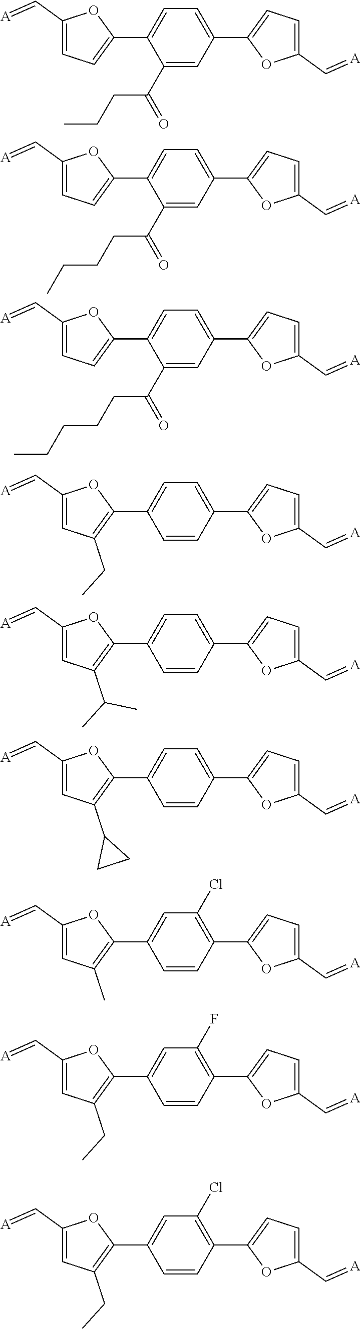

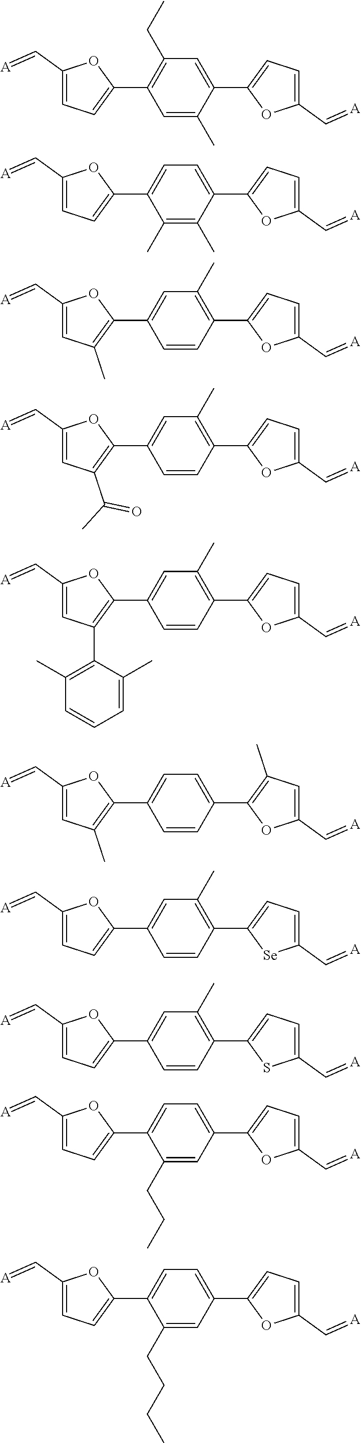

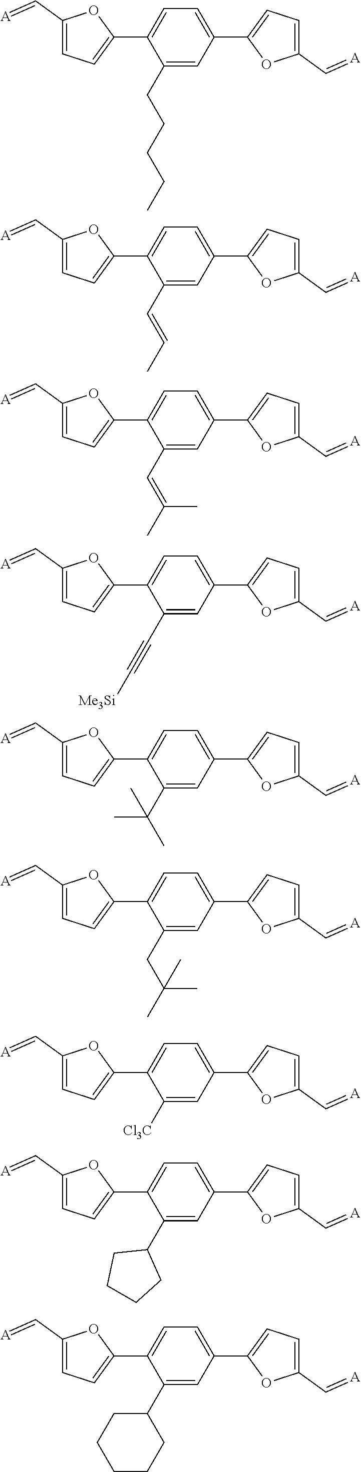

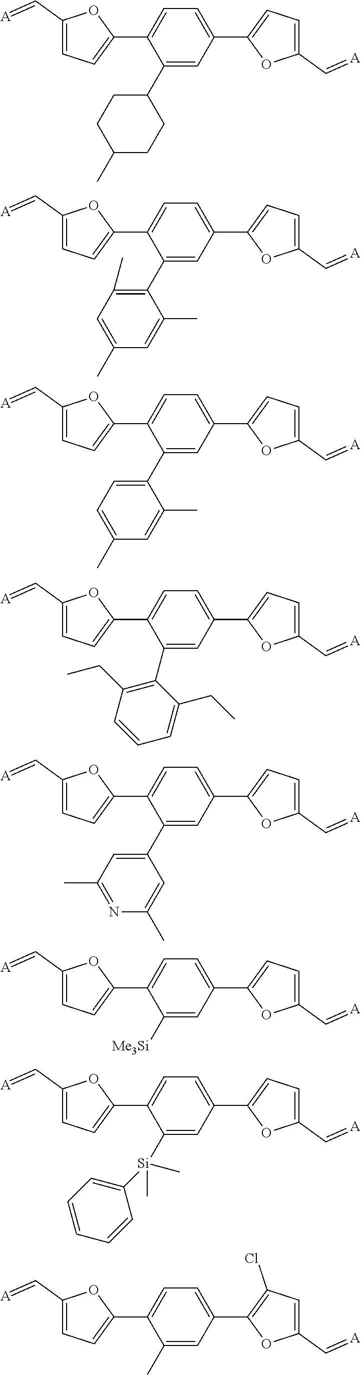

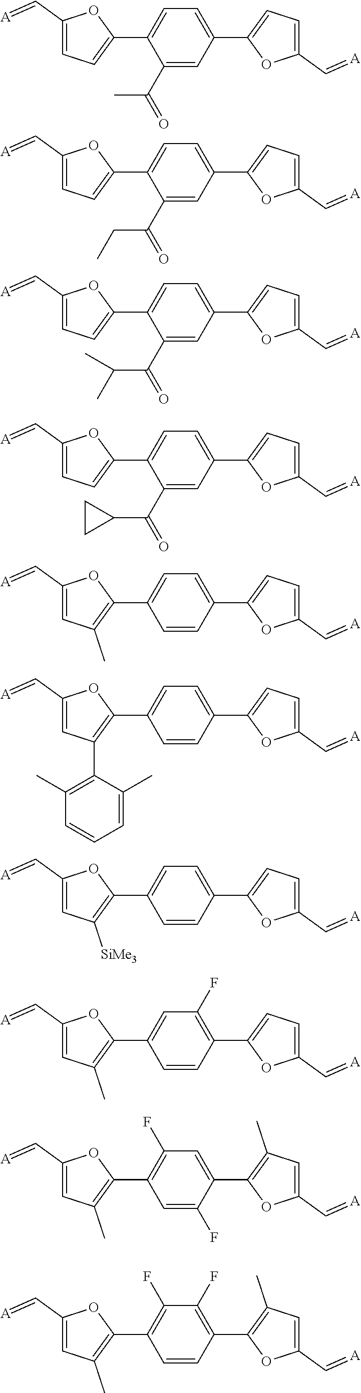

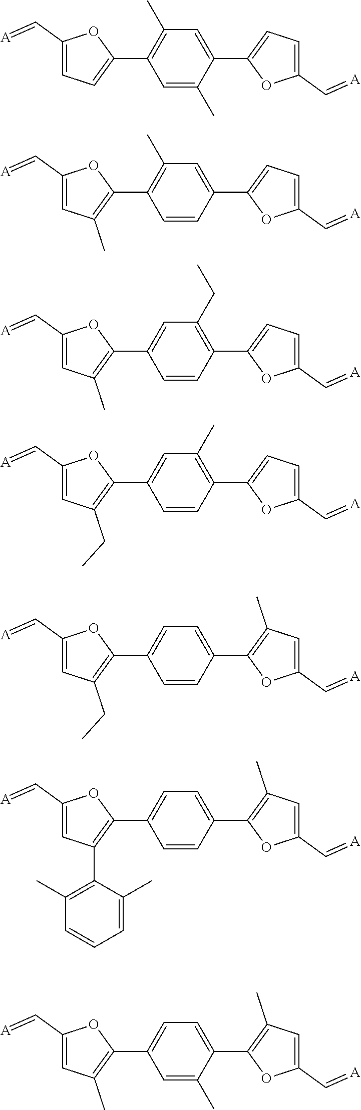

A in the specific compounds exemplified above is represented by any of the following groups.

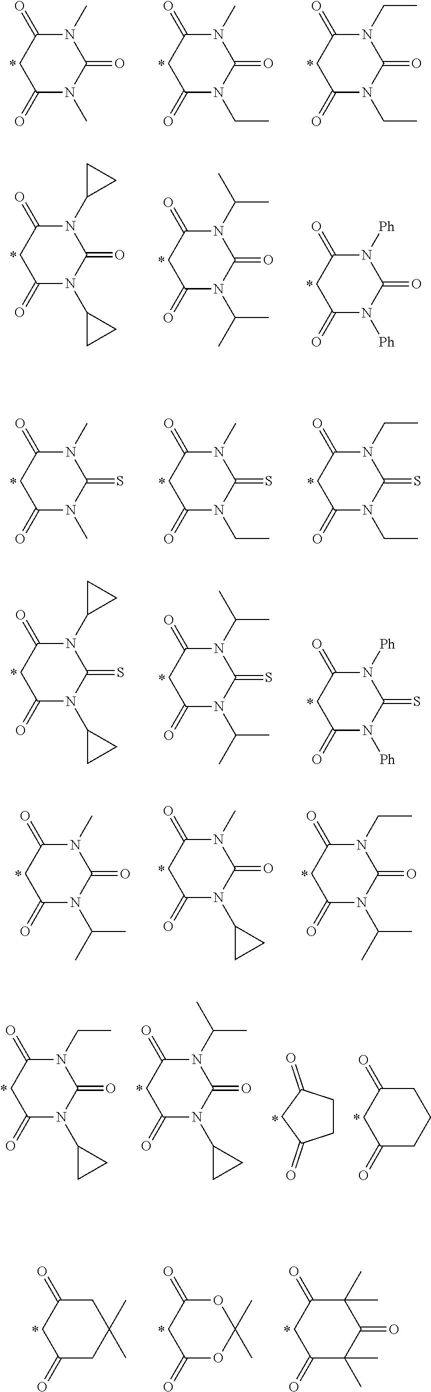

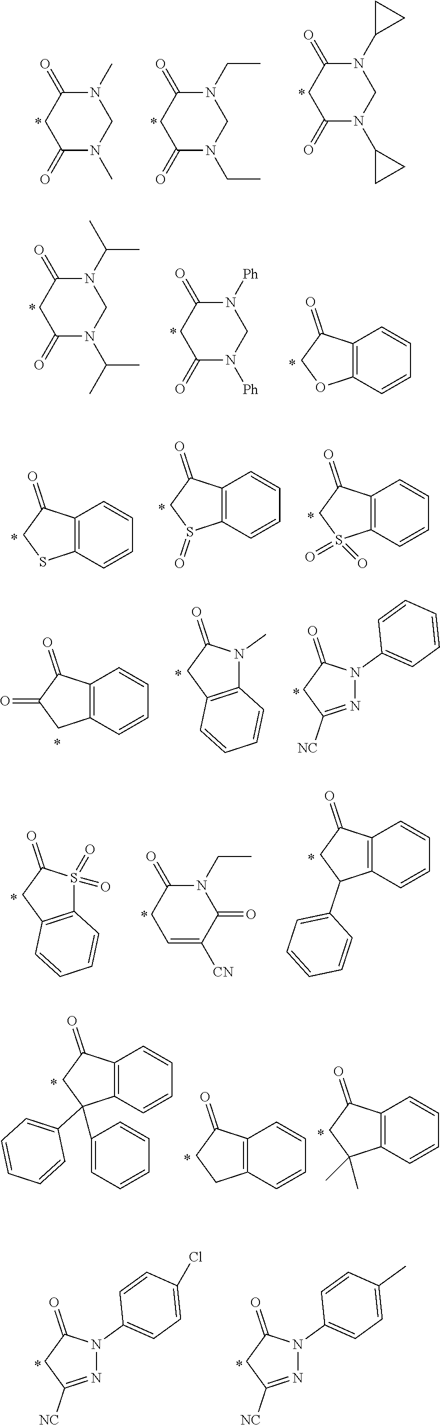

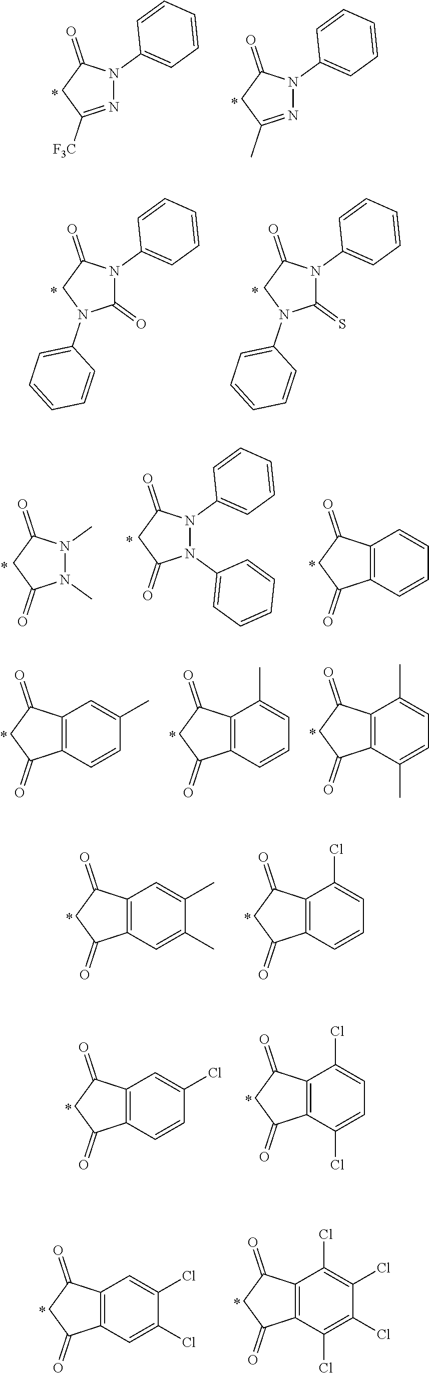

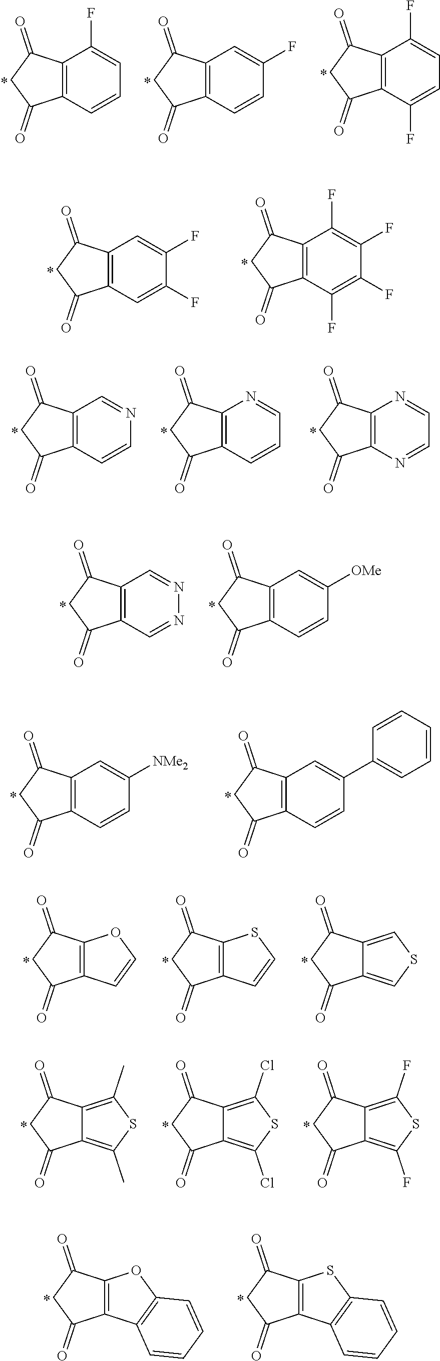

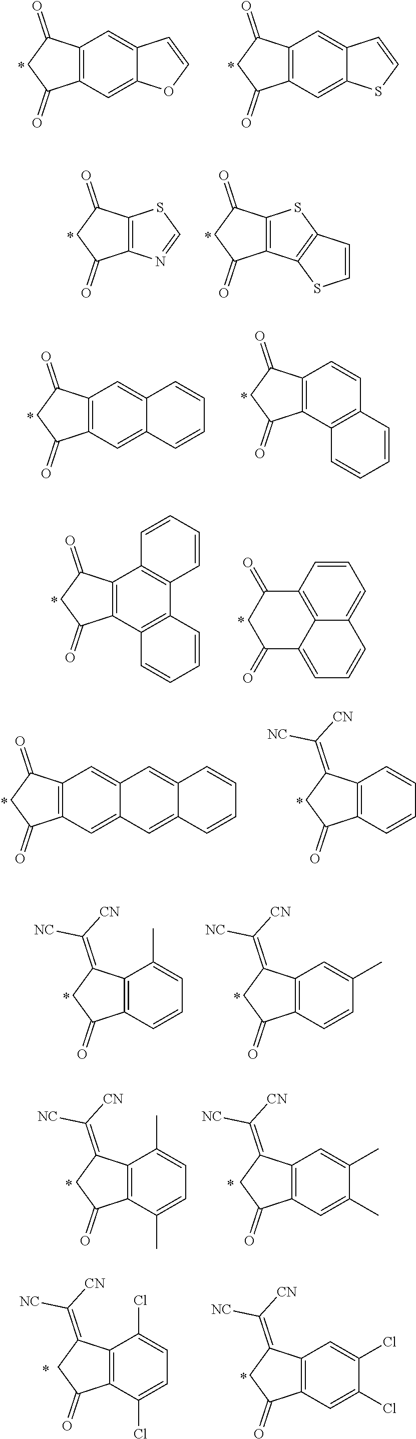

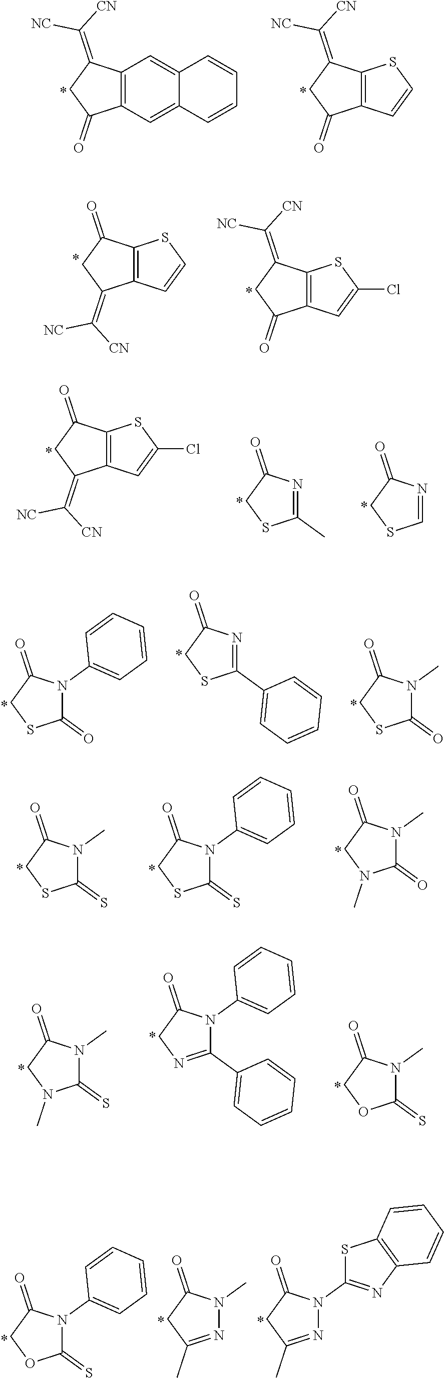

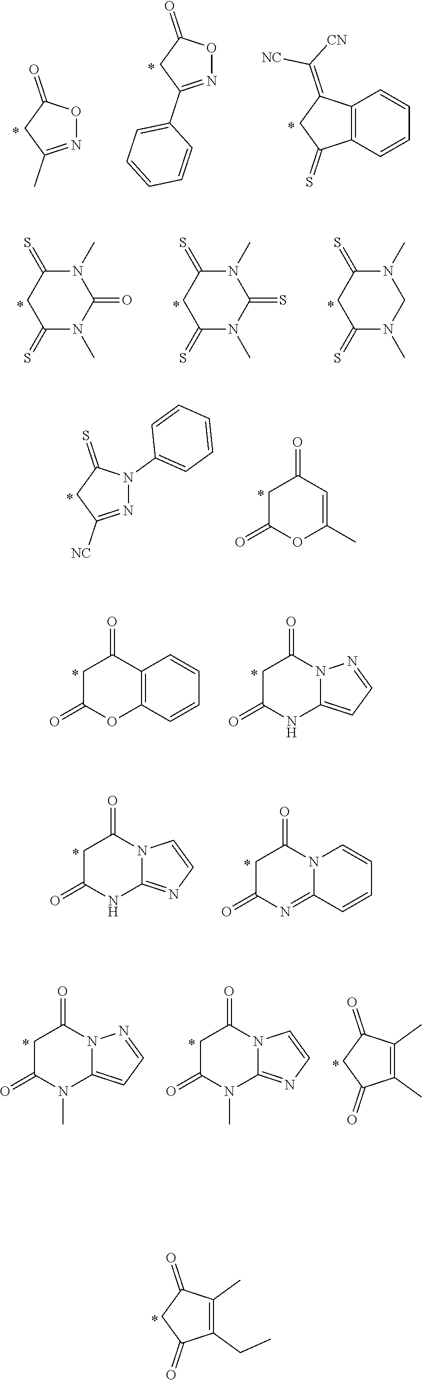

As shown in the following examples, A specified in each specific compound may be the same or different from each other.

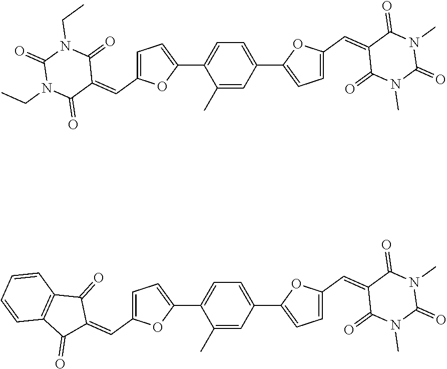 [SMILES: CCN1C(=O)C(=Cc2ccc(-c3ccc(-c4ccc(C=C5C(=O)N(C)C(=O)N(C)C5=O)o4)cc3C)o2)C(=O)N(CC)C1=O ; Cc1cc(-c2ccc(C=C3C(=O)N(C)C(=O)N(C)C3=O)o2)ccc1-c1ccc(C=C2C(=O)c3ccccc3C2=O)o1]

The specific compound may be purified as necessary.

Examples of a purification method of the specific compound include sublimation purification, purification using silica gel column chromatography, purification using gel permeation chromatography, re-slurry washing, re-purification by re-precipitation, purification using an adsorbent such as activated carbon, and recrystallization purification.

A content of the specific compound in the photoelectric conversion film (=Film thickness of specific compound in terms of single layer/Film thickness of photoelectric conversion film×100) is not particularly limited, but is preferably 15% to 75% by volume, more preferably 20% to 60% by volume, and still more preferably 20% to 50% by volume.

The specific compound may be used alone or in combination of two or more types thereof. In a case where two or more types thereof are used, it is preferable that the total amount thereof is within the above-described range.

<n-Type Organic Semiconductor>

The photoelectric conversion film preferably contains an n-type organic semiconductor, in addition to the specific compound.

The n-type organic semiconductor is a compound different from the above-described specific compound.

The n-type organic semiconductor is an acceptor-type organic semiconductor material (compound), and refers to an organic compound having a property of easily accepting an electron. That is, the n-type organic semiconductor refers to an organic compound having a larger electron affinity in a case where two organic compounds used in contact with each other. That is, any organic compound having an electron accepting property can be used as the acceptor-type organic semiconductor.

Examples of the n-type organic semiconductor include fullerenes selected from the group consisting of a fullerene and derivatives thereof; fused aromatic carbocyclic compounds (for example, a naphthalene derivative, an anthracene derivative, a phenanthrene derivative, a tetracene derivative, a pyrene derivative, a perylene derivative, a fluoranthene derivative, and the like); heterocyclic compounds with a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, thiazole, and the like); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivatives and oxadiazole derivatives; anthraquinodimethane derivatives; diphenylquinone derivatives; bathocuproine, bathophenanthroline, and derivatives thereof; triazole compounds; distyrylarylene derivatives; metal complexes having a nitrogen-containing heterocyclic compound as a ligand; silole compounds; and compounds described in paragraphs [0056] and [0057] of JP2006-100767A.

The n-type organic semiconductor (compound) is preferably fullerenes selected from the group consisting of a fullerene and derivatives thereof.

Examples of the fullerene include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the derivatives of fullerene include compounds in which a substituent is added to the above-described fullerenes. The above-described substituent is preferably an alkyl group, an aryl group, or a heterocyclic group. As the derivatives of fullerene, compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

A molecular weight of the n-type organic semiconductor is preferably 200 to 1,200 and more preferably 200 to 900.

A maximal absorption wavelength of the n-type organic semiconductor is preferably in a wavelength of 400 nm or less or in a wavelength range of 400 to 600 nm.

It is preferable that the photoelectric conversion film has a bulk heterojunction structure formed in a state in which the specific compound and the n-type organic semiconductor are mixed with each other. The bulk heterojunction structure refers to a layer in which the specific compound and the n-type organic semiconductor are mixed and dispersed in the photoelectric conversion film. The photoelectric conversion film having the bulk heterojunction structure can be formed by a wet method or a dry method. The bulk heterojunction structure is described in detail in paragraphs [0013] and [0014] of JP2005-303266A.

A difference in electron affinity between the specific compound and the n-type organic semiconductor is preferably 0.1 eV or more.

The n-type organic semiconductor may be used alone or in combination of two or more types thereof.

In a case where the photoelectric conversion film contains the n-type organic semiconductor, a content of the n-type organic semiconductor in the photoelectric conversion film (Film thickness of n-type organic semiconductor in terms of single layer/Film thickness of photoelectric conversion film×100) is preferably 15% to 75% by volume, more preferably 20% to 60% by volume, and still more preferably 20% to 50% by volume.

In a case where the n-type organic semiconductor includes fullerenes, a content of the fullerenes to the total content of the n-type organic semiconductor (Film thickness of fullerenes in terms of single layer/Total film thickness of n-type organic semiconductors in terms of single layer×100) is preferably 50% to 100% by volume, and more preferably 80% to 100% by volume. The fullerenes may be used alone or in combination of two or more types thereof.

From the viewpoint of response speed of the photoelectric conversion element, the content of the specific compound to the total content of the specific compound and the n-type organic semiconductor (Film thickness of specific compound in terms of single layer/(Film thickness of specific compound in terms of single layer+Film thickness of n-type organic semiconductor in terms of single layer)×100) is preferably 20% to 80% by volume, and more preferably 40% to 80% by volume.

In a case where the photoelectric conversion film contains the n-type organic semiconductor and a p-type organic semiconductor, the content of the specific compound (Film thickness of specific compound in terms of single layer/(Film thickness of specific compound in terms of single layer+Film thickness of n-type organic semiconductor in terms of single layer+Film thickness of p-type organic semiconductor in terms of single layer)×100) is preferably 15% to 75% by volume, and more preferably 30% to 75% by volume.

It is preferable that the photoelectric conversion film substantially contains the specific compound, the n-type organic semiconductor, and the p-type organic semiconductor contained as desired. The term “substantially” indicates that the total content of the specific compound, the n-type organic semiconductor, and the p-type organic semiconductor is 90% to 100% by mass, preferably 95% to 100% by mass, and more preferably 99% to 100% by mass with respect to the total mass of the photoelectric conversion film.

<p-Type Organic Semiconductor>

The photoelectric conversion film preferably contains a p-type organic semiconductor in addition to the above-described specific compound.

The p-type organic semiconductor is a compound different from the above-described specific compound.

The p-type organic semiconductor is a donor-type organic semiconductor material (compound), and refers to an organic compound having a property of easily donating an electron. That is, the p-type organic semiconductor refers to an organic compound having a smaller ionization potential in a case where two organic compounds are used in contact with each other.

The p-type organic semiconductor may be used alone or in combination of two or more types thereof.

Examples of the p-type organic semiconductor include triarylamine compounds (for example, N,N′-diphenyl-N,N′-bis(3-methylphenyl)-(1,1′-biphenyl)-4,4′-diamine (TPD), 4,4′-bis[N-(naphthyl)-N-Phenyl-amino]biphenyl (α-NPD), compounds described in paragraphs [0128] to [0148] of JP2011-228614A, compounds described in paragraphs [0052] to [0063] of JP2011-176259A, compounds described in paragraphs [0119] to [0158] of JP2011-225544A, compounds described in paragraphs [0044] to [0051] of JP2015-153910A, compounds described in paragraphs [0086] to [0090] of JP2012-094660A, and the like), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno[3,2-f:4,5-f′]bis[1]benzothiophene (TBBT) derivative, compounds described in paragraphs [0031] to [0036] of JP2018-014474A, compounds described in paragraphs [0043] to [0045] of WO2016/194630A, compounds described in paragraphs [0025] to [0037], and [0099] to [0109] of WO2017/159684A, compounds described in paragraphs [0029] to [0034] of JP2017-076766A, compounds described in paragraphs [0015] to [0025] of WO2018/207722A, compounds described in paragraphs [0045] to [0053] of JP2019-054228A, compounds described in paragraphs [0045] to [0055] of WO2019/058995A, compounds described in paragraphs [0063] to [0089] of WO2019/081416A, compounds described in paragraphs [0033] to [0036] of JP2019-080052A, compounds described in paragraphs [0044] to [0054] of WO2019/054125A, compounds described in paragraphs [0041] to [0046] of WO2019/093188A, compounds described in paragraphs [0034] to [0037] of JP2019-050398A, compounds described in paragraphs [0033] to [0036] of JP2018-206878A, compounds described n paragraph [0038] of JP2018-190755A, compounds described in paragraphs [0019] to [0021] of JP2018-026559A, compounds described in paragraphs [0031] to [0056] of JP2018-170487A, compounds described in paragraphs [0036] to [0041] of JP2018-078270A, compounds described in paragraphs [0055] to [0082] of JP2018-166200A, compounds described in paragraphs [0041] to [0050] of JP2018-113425A, compounds described in paragraphs [0044] to [0048] of JP2018-085430A, compounds described in paragraphs [0041] to [0045] of JP2018-056546A, compounds described in paragraphs [0042] to [0049] of JP2018-046267A, compounds described in paragraphs [0031] to [0036] of JP2018-014474A, compounds described in paragraphs [0036] to [0046] of WO2018/016465A, compounds described in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having a nitrogen-containing heterocyclic compound as a ligand.

As the p-type organic semiconductor, in addition to the above-described compounds, compounds described in JP2021-163968A, JP2022-027575A, JP2022-123944A, JP2022-122839A, JP2022-120323A, JP2022-120273A, JP2022-115832A, JP2022-108268A, JP2022-100258A, JP2022-181226A, and JP2023-005703A can also be used, and the compounds are incorporated in the present specification.

Examples of the p-type organic semiconductor also include compounds having an ionization potential smaller than that of the n-type organic semiconductor, and in a case where this condition is satisfied, the organic coloring agent exemplified as the n-type organic semiconductor can be used.

Compounds which can be used as the p-type organic semiconductor compound are exemplified below.

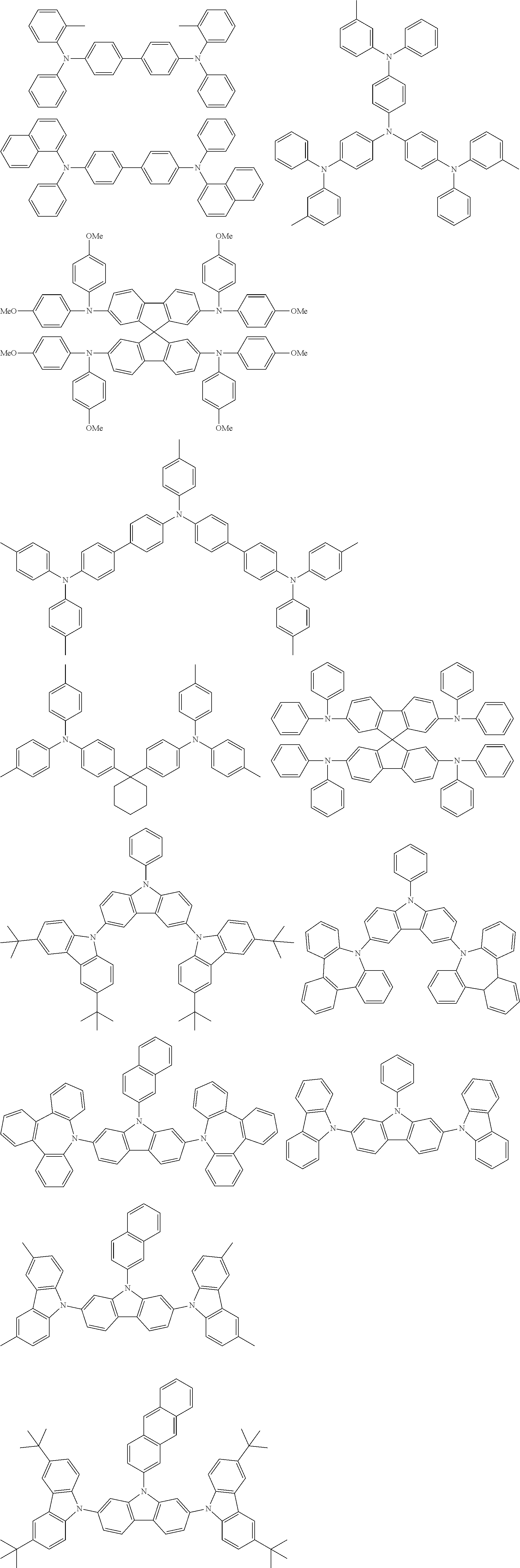

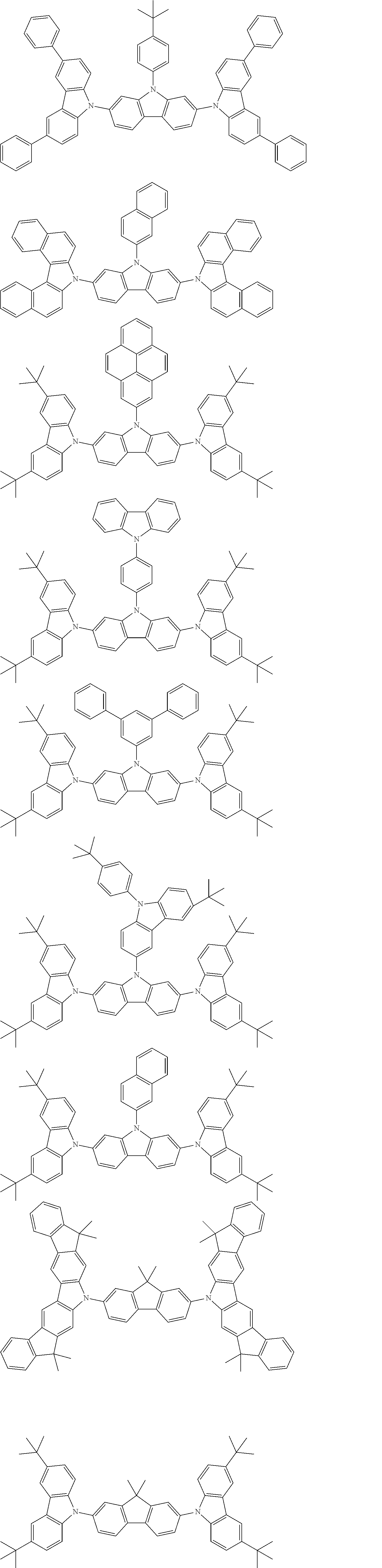

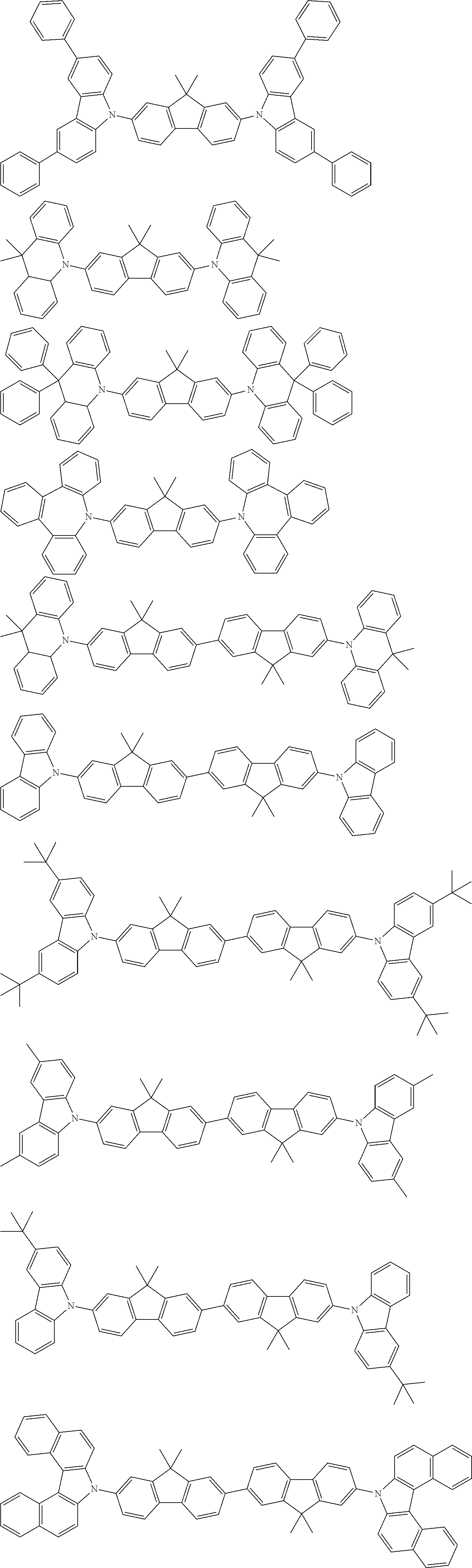

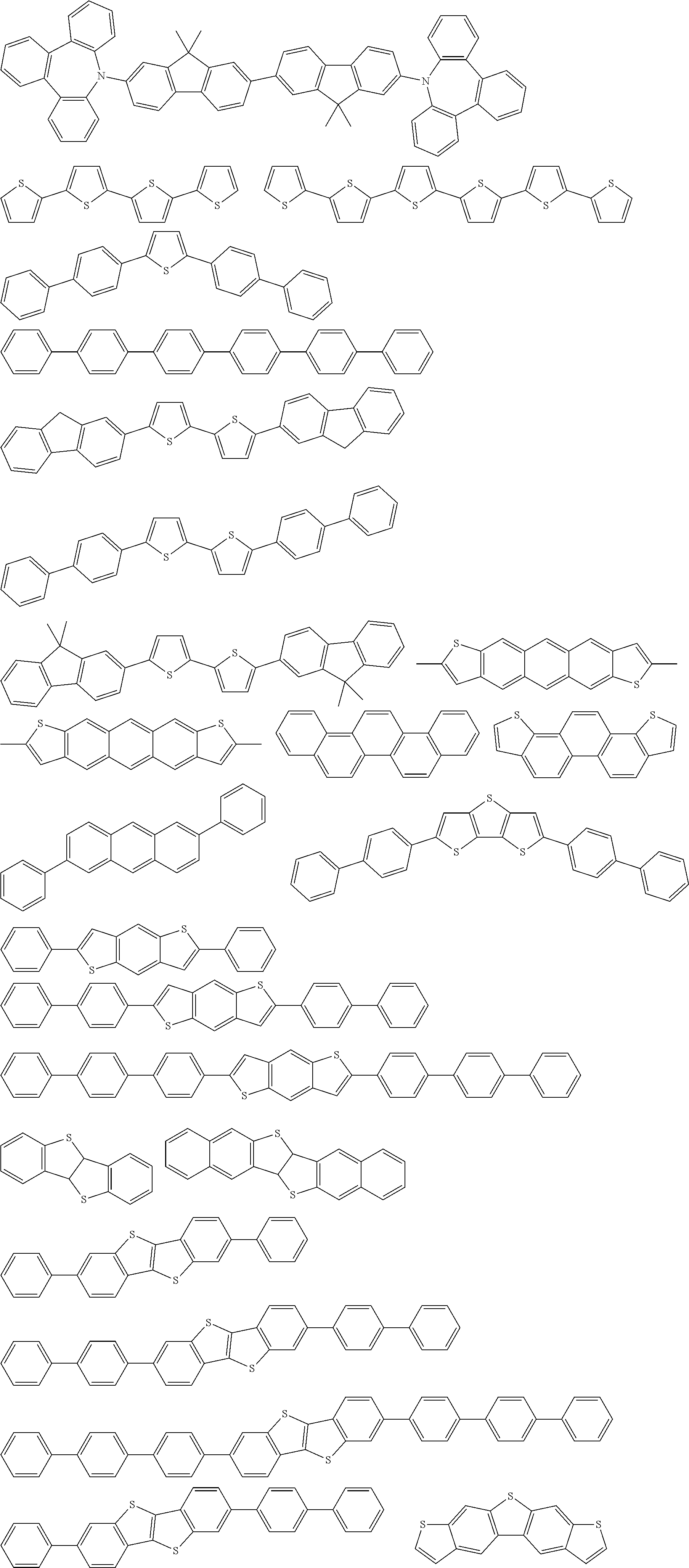

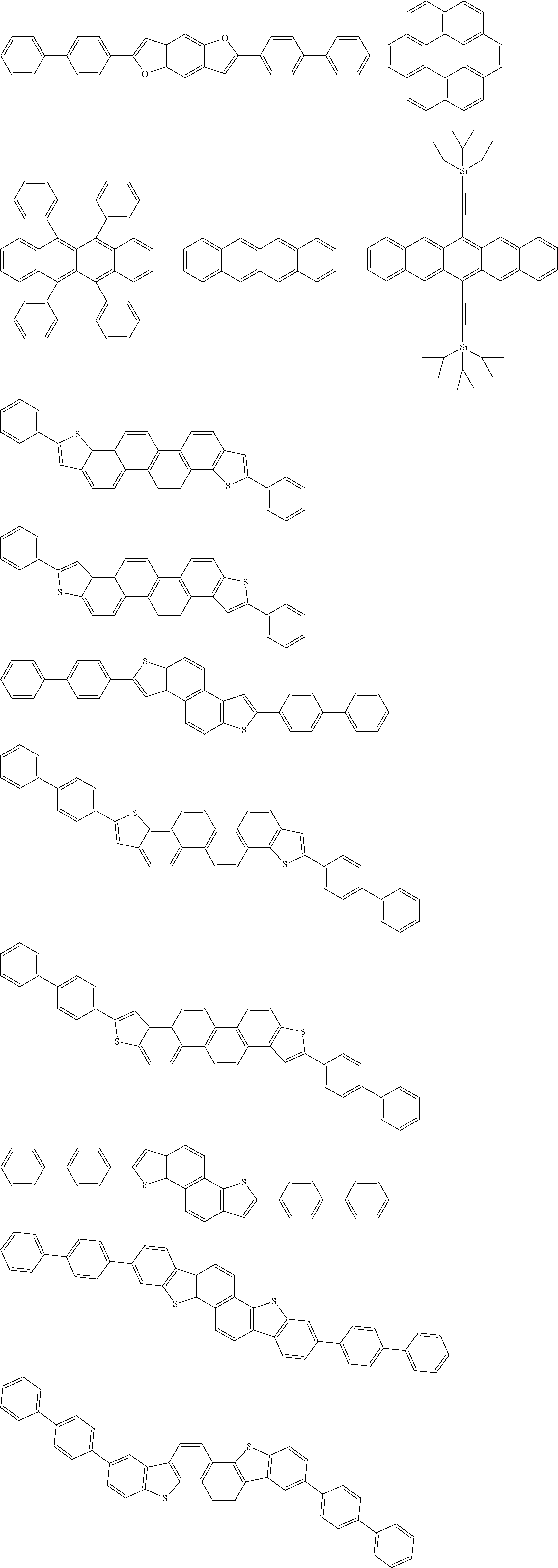

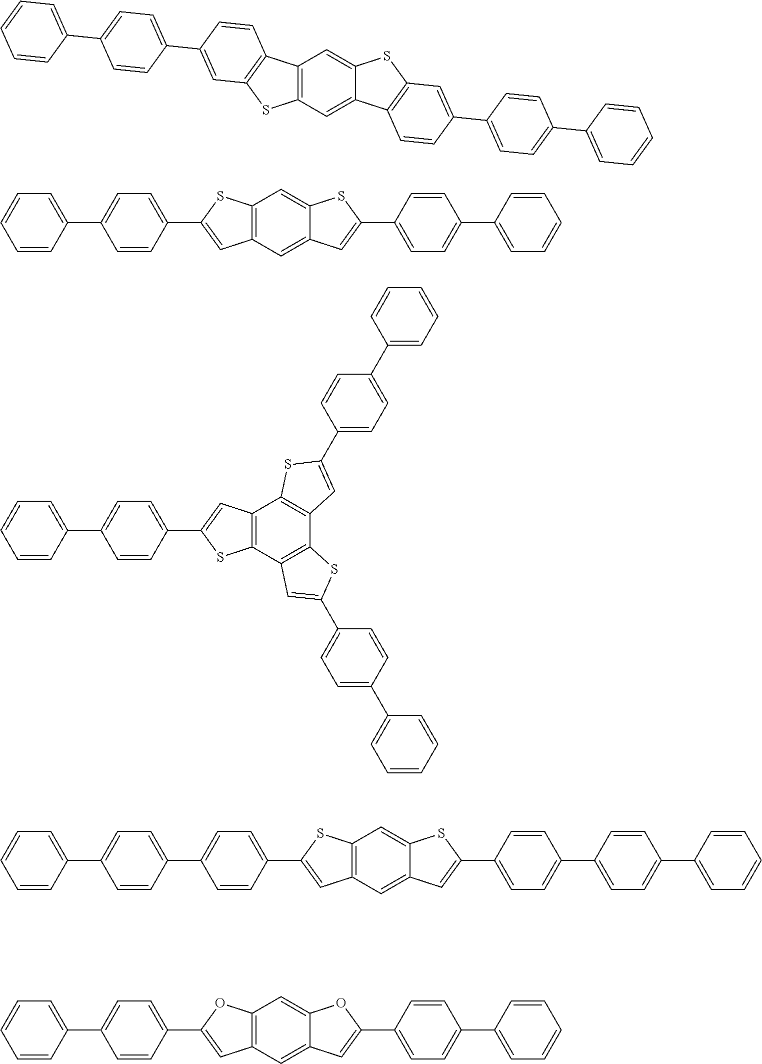

A difference in ionization potential between the specific compound and the p-type organic semiconductor is preferably 0.1 eV or more.

The p-type organic semiconductor material may be used alone or in combination of two or more types thereof.

In a case where the photoelectric conversion film contains the p-type organic semiconductor, a content of the p-type organic semiconductor in the photoelectric conversion film (Film thickness of p-type organic semiconductor in terms of single layer/Film thickness of photoelectric conversion film×100) is preferably 15% to 75% by volume, more preferably 20% to 60% by volume, and still more preferably 25% to 50% by volume.

The photoelectric conversion film containing the specific compound is a non-luminescent film, and has a feature different from an organic light emitting diode (OLED). The non-luminescent film refers to a film having a light emission quantum efficiency of 1% or less, and the light emission quantum efficiency is preferably 0.5% or less and more preferably 0.1% or less. The lower limit thereof is often 0% or more.

### <Coloring Agent>

The photoelectric conversion film may contain a coloring agent in addition to the above-described specific compound.

The coloring agent is a compound different from the above-described specific compound.

As the coloring agent, an organic coloring agent is preferable.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, a metal complex coloring agent, an imidazoquinoxaline coloring agent described in WO2020/013246A, WO2022/168856A, JP2023-010305A, and JP2023-010299A, an acceptor-donor-acceptor type coloring agent (ADA-type coloring agent) in which two acidic nuclei are bonded to a donor, and a donor-acceptor-donor type coloring agent (DAD-type coloring agent) in which two donors are bonded to an acceptor.

Among these, from the viewpoint of having a maximal absorption wavelength in a preferred range described later, and the like, the organic coloring agent is preferably a cyanine coloring agent, an imidazoquinoxaline coloring agent, or an acceptor-donor-acceptor type coloring agent.

A maximal absorption wavelength of the coloring agent is preferably in the visible light region, more preferably in a wavelength range of 400 to 650 nm, and still more preferably in a wavelength range of 450 to 650 nm.

The coloring agent may be used alone or in combination of two or more types thereof.

A content of the coloring agent with respect to the total content of the specific compound and the coloring agent in the photoelectric conversion film (=(Film thickness of coloring agent in terms of single layer/(Film thickness of specific compound in terms of single layer+Film thickness of coloring agent in terms of single layer)×100)) is preferably 15% to 75% by volume, more preferably 20% to 60% by volume, and still more preferably 20% to 50% by volume.

### <Film Formation Method>

Examples of a film formation method of the above-described photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization; and a vacuum vapor deposition method is preferable. In a case where the photoelectric conversion film is formed by the vacuum vapor deposition method, manufacturing conditions such as a degree of vacuum and a vapor deposition temperature can be set according to the conventional method.

A film thickness of the photoelectric conversion film is preferably 10 to 1,000 nm, more preferably 50 to 800 nm, and still more preferably 50 to 500 nm.

### [Electrode]

The photoelectric conversion element preferably includes an electrode.

The electrode (the upper electrode (transparent conductive film) 15 and the lower electrode (conductive film) 11) contains a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of a material constituting the upper electrode 15 include conductive metal oxides such as tin oxide doped with antimony, fluorine, or the like (antimony tin oxide (ATO) and fluorine doped tin oxide (FTO)), tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes and graphene. From the viewpoint of high conductivity and transparency, conductive metal oxides are preferable.

In general, in a case where the conductive film is thinner than a certain range, a resistance value rapidly increases in many cases. In a solid-state imaging element in which the photoelectric conversion element according to the present embodiment is incorporated, a sheet resistance may be 100 to 10,000Ω/□, and a degree of freedom of the film thickness range which can be reduced is large.

In addition, as the film thickness of the upper electrode (transparent conductive film) 15 is thinner, the amount of light which is absorbed in the upper electrode is smaller, and thus light transmittance usually increases. The increase in the light transmittance causes an increase in light absorbance in the photoelectric conversion film and an increase in the photoelectric conversion ability, which is preferable. Considering suppression of leakage current, increase in resistance value of the thin film, and increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode does not have transparency and reflects light, depending on use. Examples of a material constituting the lower electrode 11 include conductive metal oxides such as tin oxide doped with antimony, fluorine, or the like (ATO and FTO), tin oxide, zinc oxide, indium oxide, indium tin oxide (ITO), and indium zinc oxide (IZO); metals such as gold, silver, chromium, nickel, titanium, tungsten, and aluminum; conductive compounds such as oxides or nitrides of these metals (for example, titanium nitride (TiN)); mixtures or laminates of these metals and conductive metal oxides; organic conductive materials such as polyaniline, polythiophene, and polypyrrole; and carbon materials such as carbon nanotubes and graphene.

A method of forming the electrode can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the electrode material is ITO, examples thereof include an electron beam method, a sputtering method, a resistance thermal vapor deposition method, a chemical reaction method (such as a sol-gel method), and a coating method with a dispersion of indium tin oxide.

### [Charge Blocking Film: Electron Blocking Film and Hole Blocking Film]

It is preferable that the photoelectric conversion element includes one or more interlayers between the conductive film and the transparent conductive film, in addition to the photoelectric conversion film.

Examples of the above-described interlayer include a charge blocking film. In a case where the photoelectric conversion element includes the film, the characteristics (such as the quantum efficiency and the response speed) of the photoelectric conversion element to be obtained are more excellent. Examples of the charge blocking film include an electron blocking film and a hole blocking film.

### [Electron Blocking Film]

The electron blocking film is a donor-type organic semiconductor material (compound), and the above-described p-type organic semiconductor can be used.

In addition, a polymer material can also be used in the electron blocking film.

Specific examples of the polymer material include a polymer such as phenylenevinylene, fluorene, carbazole, indole, pyrene, pyrrole, picoline, thiophene, acetylene, and diacetylene, and derivatives thereof.

The electron blocking film may be configured by a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the quantum efficiency increases. Examples of the inorganic material which can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### [Hole Blocking Film]

The hole blocking film is an acceptor-type organic semiconductor material (compound), and the above-described n-type organic semiconductor described above can be used.

The hole blocking film may be configured by a plurality of films.

Examples of a method of manufacturing the charge blocking film include a dry film formation method and a wet film formation method. Examples of the dry film formation method include a vapor deposition method and a sputtering method. The vapor deposition method may be a physical vapor deposition (PVD) method or a chemical vapor deposition (CVD) method, and a physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method; and an inkjet method is preferable from the viewpoint of high-precision patterning.

Each film thickness of the charge blocking films (the electron blocking film and the hole blocking film) is preferably 3 to 200 nm, more preferably 5 to 100 nm, and still more preferably 5 to 30 nm.

### [Substrate]

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### [Sealing Layer]

The photoelectric conversion element may further include a sealing layer.

The performance of the photoelectric conversion material may deteriorate significantly due to the presence of deterioration factors such as water molecules. The deterioration can be prevented by coating and sealing the entirety of the photoelectric conversion film with a sealing layer such as diamond-like carbon (DLC) and ceramics such as metal oxide, metal nitride, or metal nitride oxide, which are dense and into which water molecules do not permeate.

Examples of the sealing layer include sealing layers described in paragraphs [0210] to [0215] of JP2011-082508A, the contents of which are incorporated herein by reference.

### [Imaging Element]

Examples of the application of the photoelectric conversion element include an imaging element.

The imaging element is an element which converts optical information of an image into the electric signal, and is usually an element in which a plurality of photoelectric conversion elements are arranged in a matrix on the same plane, optical signals are converted into electric signals in each photoelectric conversion element (a pixel), and the electric signals can be sequentially output to the outside of the imaging elements for each pixel. Therefore, each pixel is formed of one or more photoelectric conversion elements and one or more transistors.

### [Optical Sensor]

Examples of another application of the photoelectric conversion element include a photoelectric cell and an optical sensor, but it is preferable that the photoelectric conversion element according to the embodiment of the present invention is used as the optical sensor. The above-described photoelectric conversion element may be used alone as the optical sensor, or may be used as a line sensor in which the photoelectric conversion elements are linearly arranged or as a two-dimensional sensor in which the photoelectric conversion elements are arranged on a plane.

### Compound

The present invention also includes a compound. The compound according to the embodiment of the present invention is the above-described specific compound.

### EXAMPLES

Hereinbelow, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### [Materials Used for Photoelectric Conversion Film]

Various components contained in the photoelectric conversion film are shown below.

### Synthesis of Compound 1 (Synthesis Example 1 of Specific Compound)

A compound 1 was synthesized according to the following scheme.

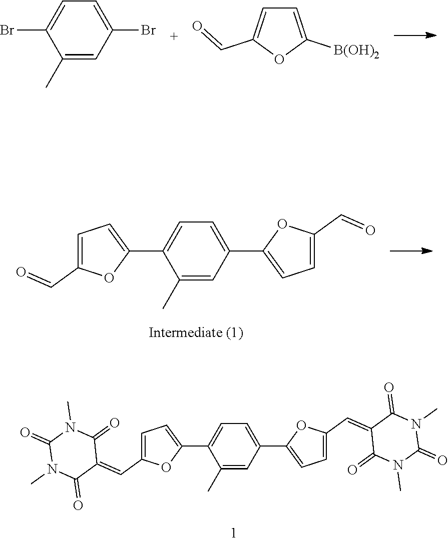

### Synthesis of Intermediate (1)

3.6 g of 2,5-dibromotoluene (manufactured by Tokyo Chemical Industry Co., Ltd.), 4.2 g of 5-formyl-2-furanboronic acid (manufactured by Tokyo Chemical Industry Co., Ltd.), 4.6 g of sodium carbonate (manufactured by FUJIFILM Wako Pure Chemical Corporation), 72 mL of 1,2-dimethoxyethane (DME, manufactured by FUJIFILM Wako Pure Chemical Corporation), 14 mL of water, and 1.0 g of bis(triphenylphosphine)palladium(II) dichloride (manufactured by FUJIFILM Wako Pure Chemical Corporation) were charged into a 300 mL three-neck flask, and the mixture was reacted at 100° C. for 5 hours under a nitrogen atmosphere. After completion of the reaction, the obtained reaction solution was filtered through Celite at room temperature, the aqueous phase was removed from the filtrate, and the obtained organic phase was washed with a saline. The obtained organic phase was dried over magnesium sulfate, the solid was separated by filtration, and the solvent was removed from the filtrate. The obtained crude product was sequentially purified by silica gel column chromatography (eluent: methylene chloride/ethyl acetate=95:5) and a gel permeation chromatography (GPC) device (eluent: chloroform) to obtain 0.72 g of an intermediate (1) (yield: 17%).

### Synthesis of Compound 1

300 mg of the intermediate (1), 435 mg of 1,3-dimethylbarbituric acid (manufactured by Tokyo Chemical Industry Co., Ltd.), 30 mL of toluene (manufactured by FUJIFILM Wako Pure Chemical Corporation), and 21 μL of piperidine (manufactured by FUJIFILM Wako Pure Chemical Corporation) were charged into a 100 mL eggplant flask, and the mixture was reacted at 100° C. for 2 hours under a nitrogen atmosphere. The precipitated solid was separated by filtration and sequentially washed with dimethylacetamide (DMAc, manufactured by FUJIFILM Wako Pure Chemical Corporation) and tetrahydrofuran (THF, manufactured by FUJIFILM Wako Pure Chemical Corporation). The obtained solid was sublimated and purified to obtain 388 mg (yield: 65%) of a compound 1. Since the compound 1 has low solubility, a structure thereof was confirmed by laser desorption ionization mass spectrometry (LDI-MS).

LDI-MS (Compound (1)): 556.1 (M⁺)

### Synthesis of Compound 2 (Synthesis Example 2 of Specific Compound)

A compound 2 was synthesized according to the following scheme.

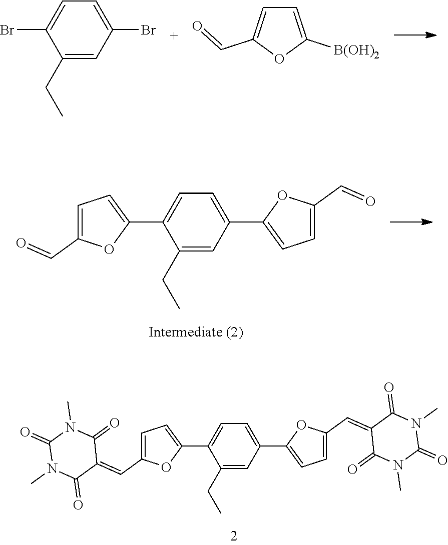

### Synthesis of Intermediate (2)

An intermediate (2) was synthesized in the same manner as the intermediate (1), except that 1,4-dibromo-2-ethylbenzene was used as a starting material instead of 2,5-dibromotoluene.

### Synthesis of Compound 2

350 mg of the intermediate (2), 483 mg of 1,3-dimethylbarbituric acid, 18 mL of toluene, and 24 μL of piperidine were charged into a 100 mL eggplant flask, and the mixture was reacted at 100° C. for 10 hours under a nitrogen atmosphere. The precipitated solid was separated by filtration and sequentially washed with DMAc and THF. The obtained solid was sublimated and purified to obtain 351 mg (yield: 52%) of a compound 2. Since the compound 2 has low solubility, a structure thereof was confirmed by LDI-MS.

LDI-MS (Compound (2)): 570.6 (M⁺)

### Synthesis of Compound 3 (Synthesis Example 3 of Specific Compound)

A compound 3 was synthesized according to the following scheme.

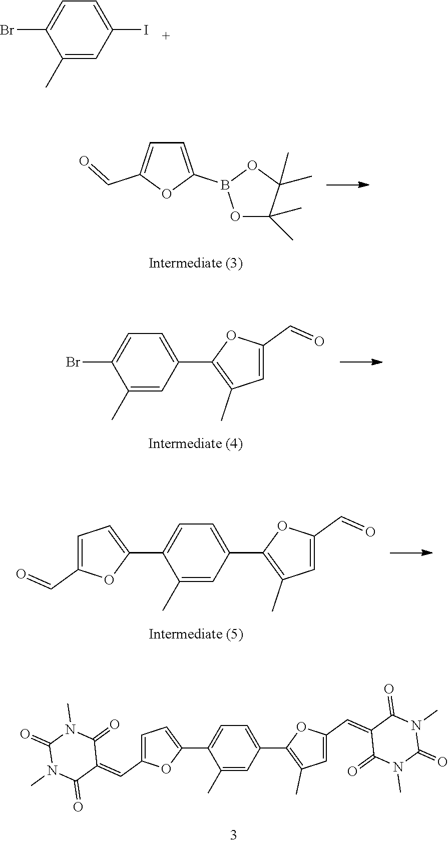

### Synthesis of Intermediate (3)

An intermediate (3) was synthesized with 4-bromo-2-furaldehyde (manufactured by Tokyo Chemical Industry Co., Ltd.) as a starting material, with reference to the methods described in the following documents.
Tetrahedron, 1990, Vol. 44, No. 4, pp. 1159 to 1210
WO2010/002933A
WO2000/032598A

### Synthesis of Intermediate (4)

1.5 g of 2-bromo-5-iodotoluene (manufactured by Tokyo Chemical Industry Co., Ltd.), 5.6 mL of a tetrahydrofuran (THF, manufactured by FUJIFILM Wako Pure Chemical Corporation) solution (1.0 M) of the intermediate (3), 1.6 g of sodium carbonate (manufactured by FUJIFILM Wako Pure Chemical Corporation), 25 mL of 1,2-dimethoxyethane (manufactured by FUJIFILM Wako Pure Chemical Corporation), 6 mL of water, and 106 mg of bis(triphenylphosphine)palladium (II) dichloride were charged into a 100 mL three-neck flask, and the mixture was reacted at 100° C. for 1.5 hours under a nitrogen atmosphere. After completion of the reaction, the obtained reaction solution was filtered through Celite at room temperature, the aqueous phase was removed from the filtrate, and the obtained organic phase was washed with a saline. The obtained organic phase was dried over magnesium sulfate, the solid was separated by filtration, and the solvent was removed from the filtrate. The obtained crude product was separated from impurities by silica gel column chromatography (eluent: hexane/ethyl acetate=8:2), and then crystallized with ethyl acetate/hexane to obtain 0.72 g (yield: 51%) of an intermediate (4).

### Synthesis of Intermediate (5)

650 mg of the intermediate (4), 360 mg of 5-formyl-2-furanboronic acid, 1.29 g of potassium carbonate (manufactured by FUJIFILM Wako Pure Chemical Corporation), 10 mL of THF, 6 mL of water, 26 mg of palladium acetate (manufactured by FUJIFILM Wako Pure Chemical Corporation), and 122 mg of tri-tert-butylphosphonium tetrafluoroborate (manufactured by FUJIFILM Wako Pure Chemical Corporation) were charged into a 100 mL three-neck flask, and the mixture was heated under reflux at 80° C. for 3.5 hours under a nitrogen atmosphere.

The obtained reaction solution was filtered through Celite at room temperature, the aqueous phase was removed from the filtrate, and the obtained organic phase was washed with a saline. The obtained organic phase was dried over magnesium sulfate, the solid was separated by filtration, and the solvent was removed from the filtrate. The crude product was separated from impurities by silica gel column chromatography (eluent: hexane/ethyl acetate=6:4), and then crystallized with methylene chloride/methanol to obtain 150 mg (yield: 22%) of an intermediate (5).

### Synthesis of Compound 3

The intermediate (5) (120 mg), 1,3-dimethylbarbituric acid (167 mg), 12 mL of toluene, and 8 μL of piperidine were charged into a 50 mL eggplant flask, and the mixture was reacted at 100° C. for 10 hours under a nitrogen atmosphere. The precipitated solid was separated by filtration and sequentially washed with DMAc and THF. The obtained solid was sublimated and purified to obtain 146 mg (yield: 62%) of a compound 3. Since the compound 3 has low solubility, a structure thereof was confirmed by LDI-MS.

LDI-MS (Compound (3)): 570.5 (M⁺)

### Synthesis of Compound 4 (Synthesis Example 4 of Specific Compound)

A compound 4 was synthesized according to the following scheme.

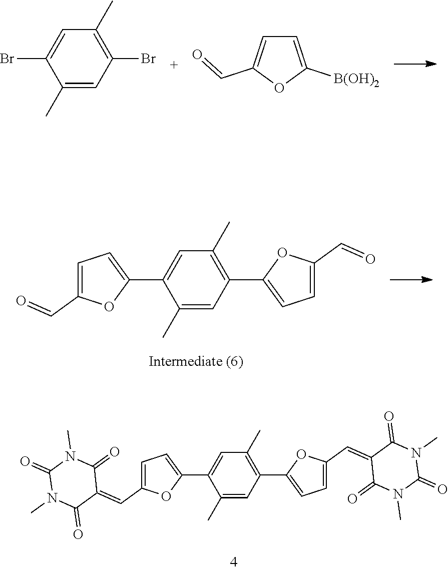

### Synthesis of Intermediate (6)

An intermediate (6) was synthesized in the same manner as the intermediate (1), except that 1,4-dibromo-2,5-dimethylbenzene was used as a starting material instead of 2,5-dibromotoluene.

### Synthesis of Compound 4

350 mg of the intermediate (6), 483 mg of 1,3-dimethylbarbituric acid, 35 mL of toluene, and 23.5 μL of piperidine were charged into a 100 mL eggplant flask, and the mixture was reacted at 100° C. for 1.5 hours under a nitrogen atmosphere. The precipitated solid was separated by filtration and sequentially washed with DMAc and THF. The obtained solid was sublimated and purified to obtain 528 mg (yield: 81%) of a compound 4.

Since the compound 4 has low solubility, a structure thereof was confirmed by LDI-MS.

LDI-MS (Compound (4)): 570.6 (M⁺)

Specific compounds and comparative compounds used in the photoelectric conversion films, other than the compounds 1 to 4, were synthesized with reference to Synthesis Examples 1 to 4 described above.

Each material used for the photoelectric conversion film is shown below. Compounds 1 to 24 correspond to the specific compounds, and Compounds C-1 to C-5 correspond to the comparative compounds.

### Specific Compound

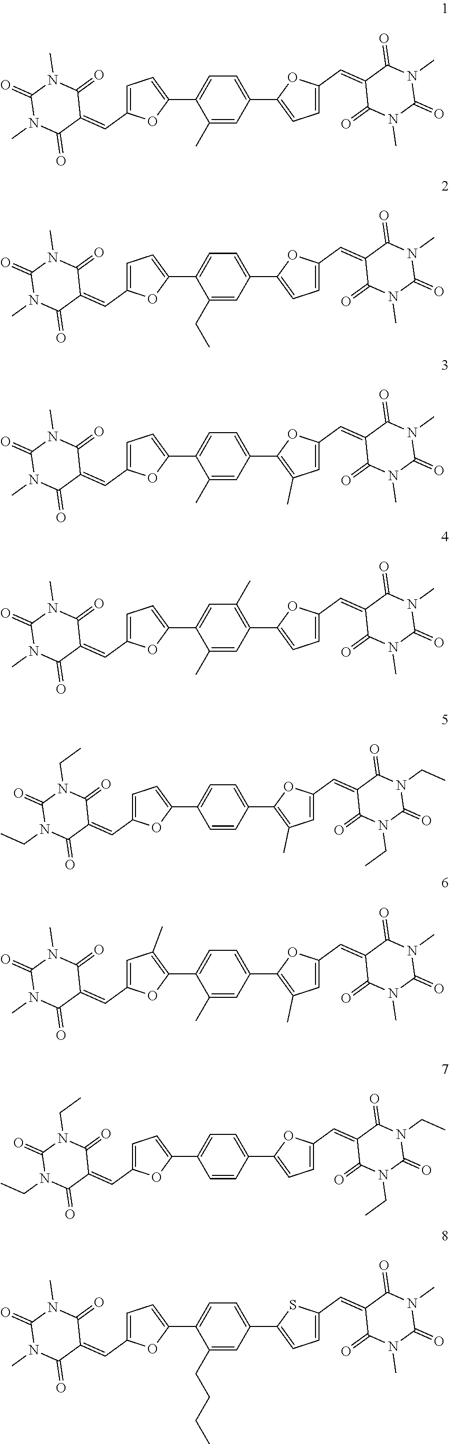

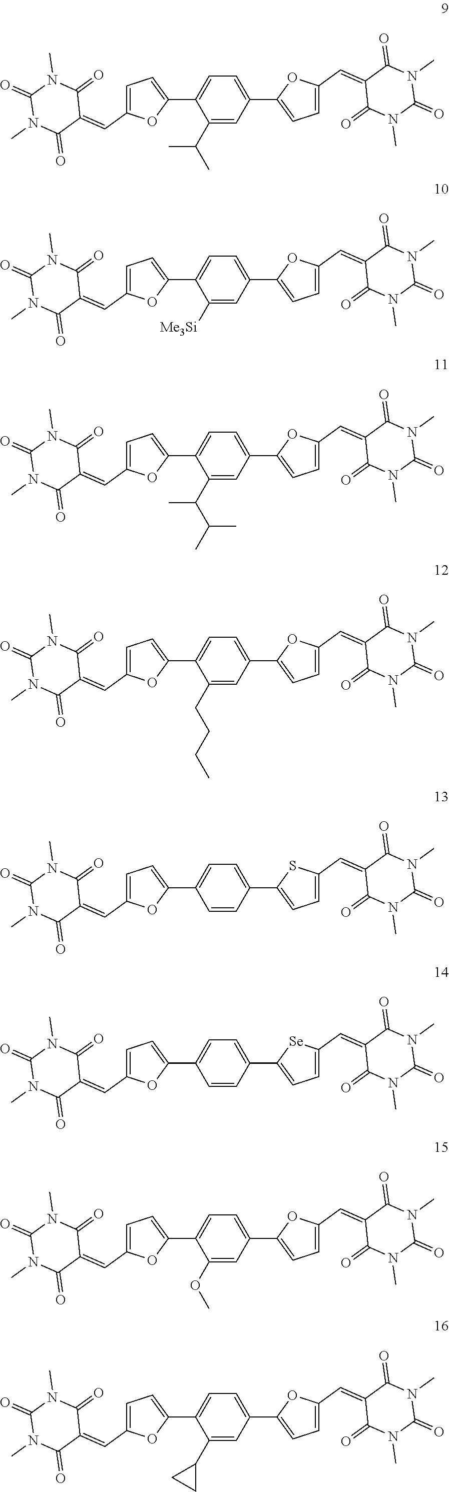

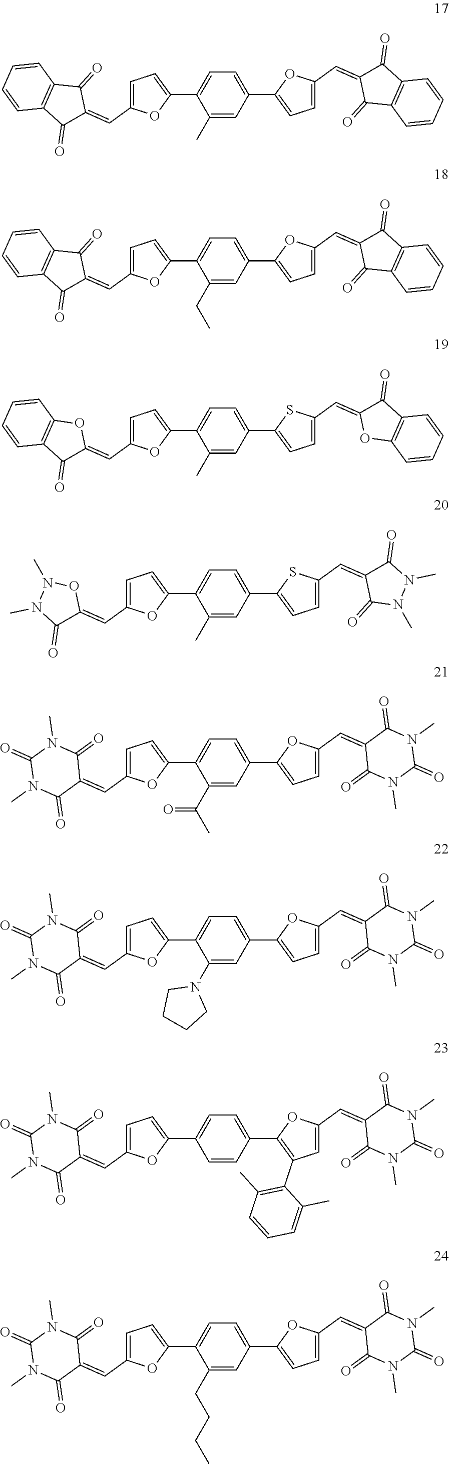

### Comparative Compound

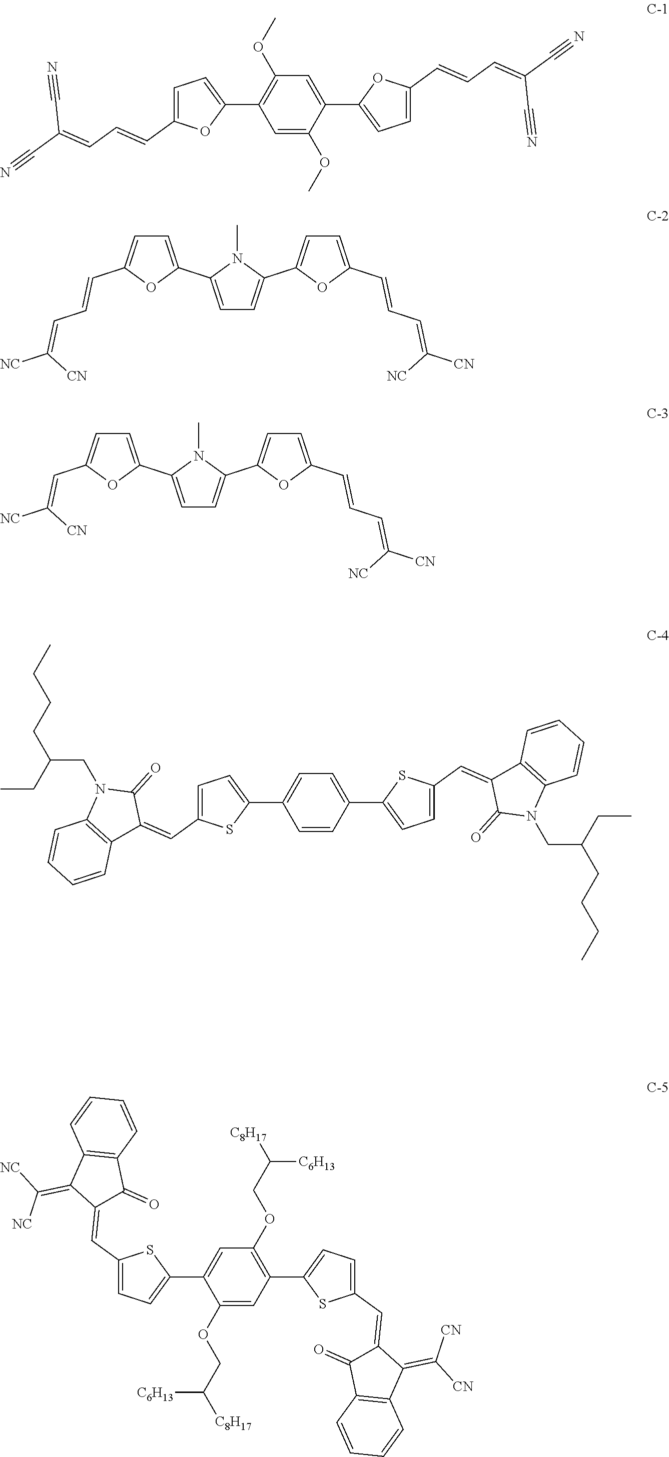

[n-Type Organic Semiconductor]
C60: fullerene (C₆₀)
   [p-Type Organic Semiconductor]

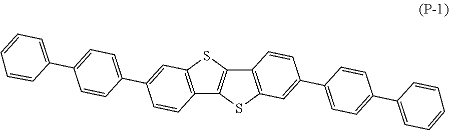 [SMILES: c1ccc(-c2ccc(-c3ccc4c(c3)sc3c5ccc(-c6ccc(-c7ccccc7)cc6)cc5sc43)cc2)cc1]

### [Coloring Agent]

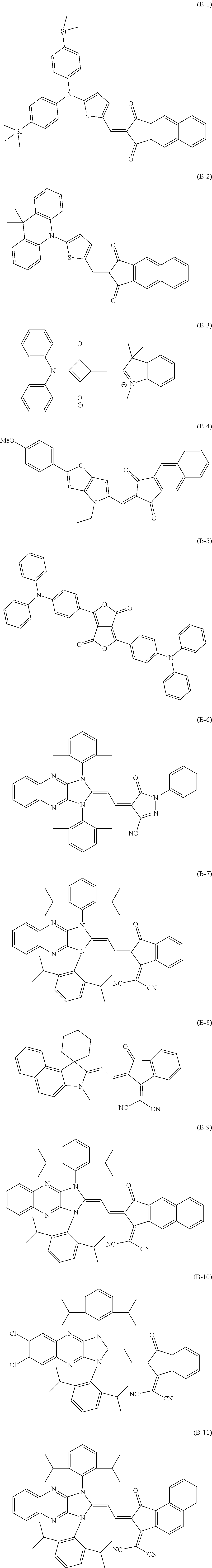

### [Evaluation]

A photoelectric conversion element was produced using the above-described materials, and Test X and Test Y were performed.

### [Test X]

### <Production of Photoelectric Conversion Element>

A photoelectric conversion element having the form of FIG. 2 was produced using the various components shown above. Here, the photoelectric conversion element included a lower electrode 11, an electron blocking film 16A, a photoelectric conversion film 12, a hole blocking film 16B, and an upper electrode 15.

Specifically, an amorphous ITO was formed into a film on a glass substrate by a sputtering method to form the lower electrode 11 (thickness: 30 nm), and the compound (EB-1) was further formed into a film on the lower electrode 11 by a vacuum thermal vapor deposition method to form the electron blocking film 16A (thickness: 30 nm).

Subsequently, in a state in which a temperature of the glass substrate was controlled to 25° C., the glass substrate was subjected to a vacuum vapor deposition method to form a film by co-vapor deposition of each specific compound or each comparative compound shown in Table 1 on the electron blocking film 16A, and the n-type organic semiconductor (fullerene (C₆₀)) and the p-type organic semiconductor (compound (P-1)) were each subjected to a co-vapor deposition method so that the thickness thereof was 80 nm in terms of single layer. As a result, the photoelectric conversion film 12 having a bulk heterojunction structure with 240 nm was formed. In this case, a film formation rate of the photoelectric conversion film 12 was set to 1.0 Å/sec.

Furthermore, the compound (EB-2) was vapor-deposited on the photoelectric conversion film 12 to form the hole blocking film 16B (thickness: 10 nm). Amorphous ITO was formed into a film on the hole blocking film 16B by a sputtering method to form the upper electrode 15 (transparent conductive film) (thickness: 10 nm). After the SiO film was formed as a sealing layer on the upper electrode 15 by a vacuum vapor deposition method, an aluminum oxide (Al₂O₃) layer was formed thereon by an atomic layer chemical vapor deposition (ALCVD) method. The obtained laminate was heated in a glove box at 150° C. for 30 minutes to obtain a photoelectric conversion element.

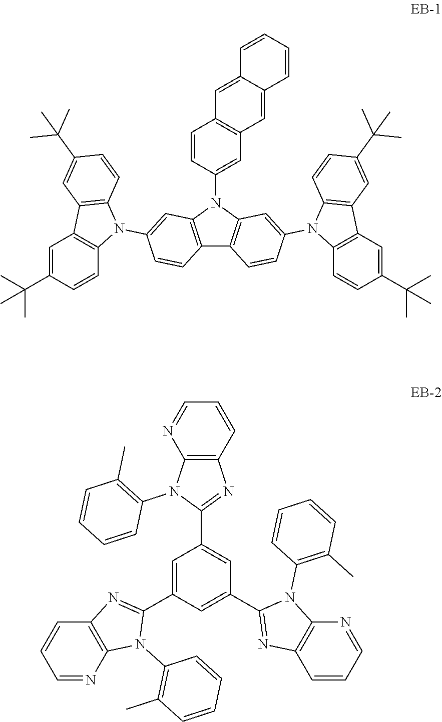 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### <Dark Current>

A dark current of each obtained photoelectric conversion element was measured by the following method. A voltage was applied to the lower electrode and the upper electrode of each of the photoelectric conversion elements with an electric field strength of 2.5×10⁵ V/cm, and a current value (dark current) in a dark place was measured. As a result, it was confirmed that all of the photoelectric conversion elements had a dark current of 50 nA/cm² or less, which indicates that all of the photoelectric conversion elements had a sufficiently low dark current.

### <Quantum Efficiency>

Quantum efficiency of each obtained photoelectric conversion element was measured by the following method. A voltage was applied to each of the photoelectric conversion elements with an electric field strength of 2.0×10⁵ V/cm, and then light was emitted from the upper electrode (transparent conductive film) side to evaluate quantum efficiency (photoelectric conversion efficiency) at a wavelength of 460 nm. The quantum efficiency was evaluated in accordance with the following standard based on a value obtained according to Expression (Si). In Formula (S1), for Examples and Comparative Examples shown in Table 1, Example 1-13 was adopted as the following reference example.

$$Quantum efficiency ( relative ratio ) = ( Quantum efficiency at wavelength of 460 nm in each of Examples or Comparative Examples ) / ( Quantum efficiency at wavelength of 460 nm in Reference Example ) \qquad Expression ( S 1 )$$

### (Evaluation Standard)

A: quantum efficiency was 1.6 or more.
B: quantum efficiency was 1.2 or more and less than 1.6.
C: quantum efficiency was 0.8 or more and less than 1.2.
D: quantum efficiency was 0.4 or more and less than 0.8.
E: quantum efficiency was less than 0.4.

### <Response Speed (Responsiveness)>

A response speed of each obtained photoelectric conversion element was evaluated by the following method. A voltage was applied to the photoelectric conversion element with an electric field strength of 2.0×10⁵ V/cm. Thereafter, a light emitting diode (LED) was turned on for an instant to emit light from the upper electrode (transparent conductive film) side, a photocurrent at this time at a wavelength of 460 nm was measured with an oscilloscope, a rise time until the signal intensity rose from 0% to 97% was measured, and the relative response speed was evaluated in accordance with the following standard based on a value obtained according to Expression (S2). In Formula (S2), for Examples and Comparative Examples shown in Table 1, Example 1-13 was adopted as the following reference example.

$$Relative response speed = ( Rise time at wavelength of 460 nm in each of Examples or Comparaive Examples ) / ( Rise time at wavelength of 460 nm in Reference Example ) \qquad Expression ( S 2 )$$

### (Evaluation Standard)

A: relative response speed was less than 0.5.
B: relative response speed was 0.5 or more and less than 1.0.
C: relative response speed was 1.0 or more and less than 1.5.
D: relative response speed was 1.5 or more and less than 2.0.
E: relative response speed was 2.0 or more.

### <Electric Field Strength Dependence of Response Speed>

For each obtained photoelectric conversion element, electric field strength dependence of the response speed was evaluated by the following method.

A response speed at 7.5×10⁴ V/cm was measured by the same procedure as in the evaluation of <Response speed> above, except that the voltage applied to each photoelectric conversion element was changed to 7.5×10⁴ V/cm; and the electric field strength dependence of the response speed was evaluated in accordance with the following standard based on a value obtained according to Expression (S3).

In Expression (S3), each photoelectric conversion element used for the numerator and the denominator are the same. For example, with regard to Example 1-1, the rise time of the photoelectric conversion efficiency at 7.5×10⁴ V/cm and a wavelength of 460 nm in Example 1-1 and the rise time of the photoelectric conversion efficiency at 2.0×10⁵ V/cm and a wavelength of 460 nm in Example 1-1 were compared.

$$Electric field strength dependence of response speed = ( Rise time at 7.5 \times {10}^{4} V / cm at wavelength of 460 nm in each of Examples or Comparative Examples ) / ( Rise time at 2. \times 1 {0}^{5} V / cm at wavelength of 460 nm in each of Examples or Comparative Examples ) \qquad Expression ( S 3 )$$

### (Evaluation Standard)

A: electric field strength dependence of the response speed was less than 2.0.
B: electric field strength dependence of the response speed was 2.0 or more and less than 3.0.
C: electric field strength dependence of the response speed was 3.0 or more and less than 4.0.
D: electric field strength dependence of the response speed was 4.0 or more and less than 5.0.
E: electric field strength dependence of the response speed was 5.0 or more.

### <Manufacturing Suitability>

Manufacturing suitability of each obtained photoelectric conversion element was evaluated by the following method. A photoelectric conversion element of each of Examples or each of Comparative Examples was produced as by the same procedure as <Production of photoelectric conversion element> above, except that the film formation rate of the photoelectric conversion film 12 was changed to 3.0 Å/sec.

The photoelectric conversion element obtained in <Production of photoelectric conversion element> above was defined as a photoelectric conversion element (A), the photoelectric conversion element obtained by setting the film formation rate of the photoelectric conversion film 12 to 3.0 Å/sec was defined as a photoelectric conversion element (B), and each quantum efficiency was determined as by the same procedure as in the evaluation of <Quantum efficiency> above. For the photoelectric conversion element having the same configuration of Examples or Comparative Examples, a relative ratio B/A of the quantum efficiency of the photoelectric conversion element (B) to the quantum efficiency of the photoelectric conversion element (A) (Quantum efficiency of photoelectric conversion element (B)/Quantum efficiency of photoelectric conversion element (A)) was calculated, and the manufacturing suitability was evaluated according to the following standard using the obtained value.

### (Evaluation Standard)

A: relative ratio B/A was 0.90 or more.
B: relative ratio B/A was 0.85 or more and less than 0.90.
C: relative ratio B/A was 0.80 or more and less than 0.85.
D: relative ratio B/A was 0.75 or more and less than 0.80.
E: relative ratio B/A was less than 0.75.

Table 1 shows the evaluation results of Test X.

The respective notations in Table 1 indicate the following.

In the column of “R^{Z2}”, a case where at least one of Z¹, . . . , or Z⁸ in Formula (1) was represented by —CR^{Z2}═ is indicated as “A”, and a case other than the above case is indicated as “B”.

In the column of “R^{Z2} type limitation”, a case where the group represented by R^{Z2} represented a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from the substituent group S, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃ is indicated as “A”, and a case other than the above case is indicated as “B”.

In the column of “R^{Z2} type limitation 2”, a case where the group represented by R^{Z2} represented a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, an acyl group having 2 or 3 carbon atoms, an aromatic ring group which may have a substituent selected from the substituent group T, and an aliphatic heterocyclic group which may have a substituent selected from the substituent group T is indicated as “A”, and a case other than the above case is indicated as “B”.

In the column of “Formula (C-1) or Formula (C-2)”, a case where the group represented by Formula (A-1) in Formula (1) was the group represented by Formula (C-1) or the group represented by Formula (C-2) is indicated as “A”, and a case other than the above case is indicated as “B”.

In the column of “Formula (C-2)”, a case where the group represented by Formula (A-1) was the group represented by Formula (C-2) is indicated as “A”, and a case other than the above case is indicated as “B”.

In the column of “Furan”, a case where X¹ in Formula (1) was an oxygen atom is indicated as “A”, and a case other than the above case is indicated as “B”.

In addition, the description of “Not vapor-depositable” indicates that the compound C-5 could not be vapor-deposited as a result of the evaluation results of Comparative Example 1-5, in a case where the formation of the photoelectric conversion film was attempted in the production of the photoelectric conversion element.

TABLE 1

| | | | | | | | | | | Electric field | |
|---|---|---|---|---|---|---|---|---|---|---|---|
| | | | | | Formula | | | | | strength | |
| | | | | | (C-1) or | | | | | dependence | |
| | | | R^{Z2} type | R^{Z2} type | Formula | Formula | | Quantum | Response | of response | Manufacturing |
| | Compound | R^{Z2} | limitation | limitation 2 | (C-2) | (C-2) | Furan | efficiency | speed | speed | suitability |
| Example 1-1 | 1 | A | A | A | A | A | A | A | A | A | A |
| Example 1-2 | 2 | A | A | A | A | A | A | A | A | A | A |
| Example 1-3 | 3 | A | A | A | A | A | A | A | A | A | A |
| Example 1-4 | 4 | A | A | A | A | A | A | A | A | A | A |
| Example 1-5 | 5 | A | A | A | A | A | A | A | A | A | A |
| Example 1-6 | 6 | A | A | A | A | A | A | A | A | A | A |
| Example 1-7 | 7 | B | — | — | A | A | A | A | B | B | B |
| Example 1-8 | 8 | A | B | — | A | A | B | C | B | B | C |
| Example 1-9 | 9 | A | A | A | A | A | A | A | A | A | A |
| Example 1-10 | 10 | A | A | B | A | A | A | A | A | A | B |
| Example 1-11 | 11 | A | A | B | A | A | A | A | A | A | B |
| Example 1-12 | 12 | A | A | B | A | A | A | A | A | A | B |
| Example 1-13 | 13 | B | — | — | A | A | B | C | C | B | C |
| Example 1-14 | 14 | B | — | — | A | A | B | C | C | B | C |
| Example 1-15 | 15 | A | A | B | A | A | A | A | A | A | B |
| Example 1-16 | 16 | A | A | A | A | A | A | A | A | A | A |
| Example 1-17 | 17 | A | A | A | A | B | A | B | A | B | A |
| Example 1-18 | 18 | A | A | A | A | B | A | B | A | B | A |
| Example 1-19 | 19 | A | A | A | B | — | B | C | C | C | C |
| Example 1-20 | 20 | A | A | A | B | — | B | C | C | C | C |
| Example 1-21 | 21 | A | A | A | A | A | A | A | A | A | A |
| Example 1-22 | 22 | A | A | A | A | A | A | A | A | A | A |
| Example 1-23 | 23 | A | A | A | A | A | A | A | A | A | A |
| Example 1-24 | 24 | A | B | — | A | A | A | A | B | A | B |
| Comparative | C-1 | — | — | — | — | — | — | D | E | D | D |
| Example 1-1 | | | | | | | | | | | |
| Comparative | C-2 | — | — | — | — | — | — | E | E | D | D |
| Example 1-2 | | | | | | | | | | | |
| Comparative | C-3 | — | — | — | — | — | — | E | E | D | D |
| Example 1-3 | | | | | | | | | | | |
| Comparative | C-4 | — | — | — | — | — | — | E | D | E | E |
| Example 1-4 | | | | | | | | | | | |
| Comparative | C-5 | — | — | — | — | — | — | Not vapor-depositable | | | |
| Example 1-5 | | | | | | | | | | | |

As is clear from the results in Table 1, it was found that the photoelectric conversion elements of Examples according to the present invention had excellent quantum efficiency.

On the other hand, the photoelectric conversion elements of Comparative Example, using the comparative compound not corresponding to the specific compound, had an insufficient quantum efficiency.

In addition, from the results in Table 1, it was found that, in a case where at least one of Z¹, . . . , or Z⁸ in Formula (1) was represented by —CR^{Z2}═, the electric field strength dependence of the response speed was more excellent (comparison between Examples 1-7 and 1-24, and the like).

It was found that, in a case where the group represented by R^{Z2} represented a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from the substituent group S, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃, the response speed was more excellent (comparison between Examples 1-10 to 1-12, 1-24, and the like).

It was found that, in a case where the group represented by R^{Z2} represented a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, an acyl group having 2 or 3 carbon atoms, an aromatic ring group which may have a substituent selected from the substituent group T, and an aliphatic heterocyclic group which may have a substituent selected from the substituent group T, the manufacturing suitability was more excellent (comparison between Examples 1-1 to 1-6, 1-10 to 1-12, and the like).

It was found that, in a case where, in Formula (1), the group represented by Formula (A-1) was the group represented by Formula (C-1) or the group represented by Formula (C-2), the electric field strength dependence of the response speed was more excellent (comparison between Examples 1-13 and 1-14, 1-19 and 1-20, and the like).

It was found that, in a case where the group represented by Formula (A-1) was the group represented by Formula (C-2), the quantum efficiency and the electric field strength dependence of the response speed were more excellent (comparison of Examples 16 to 18, and the like).

It was found that, in a case where X¹ in Formula (1) was an oxygen atom, the quantum efficiency and the manufacturing suitability were more excellent (comparison between Example 1-7 and Examples 1-13 and 1-14, and the like).

### [Test Y]

### <Production of Photoelectric Conversion Element>

A photoelectric conversion film was formed by co-vapor depositing each specific compound or each comparative compound, the n-type organic semiconductor (fullerene (C60)), the p-type organic semiconductor (compound (P-1)), and the coloring agent, which were shown in Table 2, in a ratio of compound:coloring agent:p-type organic semiconductor:n-type organic semiconductor=1:1:2:2 in terms of a single layer by a vacuum vapor deposition method, and a photoelectric conversion element of each of Examples and each of Comparative Examples was produced in the same manner for the other procedures as in Test X.

### <Dark Current>

The dark current was measured in the same manner as in Test X. As a result, it was confirmed that all of the photoelectric conversion elements had a dark current of 50 nA/cm² or less, which indicates that all of the photoelectric conversion elements had a sufficiently low dark current.

### <Quantum Efficiency>

Quantum efficiency of each obtained photoelectric conversion element was measured by the following method. A voltage was applied to each of the photoelectric conversion elements with an electric field strength of 2.0×10⁵ V/cm, and then light was emitted from the upper electrode (transparent conductive film) side to evaluate quantum efficiency at a wavelength of 460 nm or at a wavelength of 600 nm. The quantum efficiency was evaluated in accordance with the following evaluation standard based on a value obtained according to Expression (S4).

In Expression (S4), the quantum efficiencies of the denominator and the numerator are the quantum efficiencies at the same wavelength. In addition, for Examples and Comparative Examples shown in Table 2, Examples 2-18 was adopted as the following reference example.

$$Quantum efficiency ( relative ratio ) = ( Quantum efficiency at wavelength of 460 nm or at wavelength of 600 nm in each of Examples or Comparative Examples ) / ( Quantum efficiency at wavelength of 460 nm or at wavelength of 600 nm in Reference Example ) \qquad Expression ( S 4 )$$

### (Evaluation Standard)

Evaluation standards for the quantum efficiency at a wavelength of 460 nm and a wavelength of 600 nm are as follows.
A: quantum efficiency was 1.6 or more.
B: quantum efficiency was 1.2 or more and less than 1.6.
C: quantum efficiency was 0.8 or more and less than 1.2.
D: quantum efficiency was 0.4 or more and less than 0.8.
E: quantum efficiency was less than 0.4.

### <Response Speed>

A response speed of each obtained photoelectric conversion element was evaluated by the following method. A voltage was applied to the photoelectric conversion element with an electric field strength of 2.0×10⁵ V/cm. Thereafter, LED was turned on for an instant to emit light from the upper electrode (transparent conductive film) side, a photocurrent at this time at a wavelength of 460 nm or at a wavelength of 600 nm was measured with an oscilloscope, a rise time until the signal intensity rose from 0% to 97% was measured, and the relative response speed was evaluated in accordance with the following standard based on a value obtained according to Expression (S5).

In Expression (S5), the rise time of the denominator and the rise time of the numerator are the rise time at the same wavelength. In addition, for Examples and Comparative Examples shown in Table 2, Examples 2-18 was adopted as the following reference example.

$$Relative response speed = ( Rise time at wavelength of 460 nm or at wavelength of 600 nm in each of Examples or Comparative Examples ) / ( Rise time at wavelength of 460 nm or at wavelength of 600 nm in Reference Example ) \qquad Expression ( S 5 )$$

### (Evaluation Standard)

A: relative response speed was less than 0.5.
B: relative response speed was 0.5 or more and less than 1.0.
C: relative response speed was 1.0 or more and less than 1.5.
D: relative response speed was 1.5 or more and less than 2.0.
E: relative response speed was 2.0 or more.

### <Electric Field Strength Dependence of Response Speed>

For each obtained photoelectric conversion element, electric field strength dependence of the response speed was evaluated by the following method.

A response speed at 7.5×10⁴ V/cm was measured by the same procedure as in the evaluation of the response speed in Test Y, except that the voltage applied to each photoelectric conversion element was changed to 7.5×10⁴ V/cm; and the electric field strength dependence of the response speed was evaluated in accordance with the following standard based on a value obtained according to Expression (S6). In Expression (S6), the photoelectric conversion elements in the numerator and the denominator are the same at the same wavelength.

$$Electric field strength dependence of response speed = ( Rise time at 7.5 \times {10}^{4} V / cm at wavelength of 460 nm or at wavelength of 600 nm in each of Examples or Comparative Examples ) / ( Rise time at 2. \times 1 {0}^{5} V / cm at wavelength of 460 nm or at wavelength of 600 nm in each of Examples or Comparative Examples ) \qquad Expression ( S 6 )$$

### (Evaluation Standard)

A: electric field strength dependence of the response speed was less than 2.0.
B: electric field strength dependence of the response speed was 2.0 or more and less than 3.0.
C: electric field strength dependence of the response speed was 3.0 or more and less than 4.0.
D: electric field strength dependence of the response speed was 4.0 or more and less than 5.0.
E: electric field strength dependence of the response speed was 5.0 or more.

Table 2 shows the evaluation results of Test Y.

Each notation in Table 2 is as described above for each notation in Table 1.

TABLE 2

| | | | | | | Formula | | |
|---|---|---|---|---|---|---|---|---|
| | | Coloring | | | R^{Z2} type | (C-1) or | | |
| | | agent used in | | R^{Z2} type | limitation | Formula | Formula | |
| | Compound | combination | R^{Z2} | limitation | 2 | (C-2) | (C-2) | Furan |
| Example 2-1 | 1 | B-1 | A | A | A | A | A | A |
| Example 2-2 | 1 | B-2 | A | A | A | A | A | A |
| Example 2-3 | 1 | B-3 | A | A | A | A | A | A |
| Example 2-4 | 1 | B-4 | A | A | A | A | A | A |
| Example 2-5 | 1 | B-5 | A | A | A | A | A | A |
| Example 2-6 | 1 | B-6 | A | A | A | A | A | A |
| Example 2-7 | 1 | B-7 | A | A | A | A | A | A |
| Example 2-8 | 1 | B-8 | A | A | A | A | A | A |
| Example 2-9 | 1 | B-9 | A | A | A | A | A | A |
| Example 2-10 | 1 | B-10 | A | A | A | A | A | A |
| Example 2-11 | 1 | B-11 | A | A | A | A | A | A |
| Example 2-12 | 2 | B-7 | A | A | A | A | A | A |
| Example 2-13 | 3 | B-7 | A | A | A | A | A | A |
| Example 2-14 | 4 | B-7 | A | A | A | A | A | A |
| Example 2-15 | 7 | B-7 | B | — | — | A | A | A |
| Example 2-16 | 10 | B-7 | A | A | B | A | A | A |
| Example 2-17 | 12 | B-7 | A | A | B | A | A | A |
| Example 2-18 | 13 | B-7 | B | — | — | A | A | B |
| Example 2-19 | 15 | B-7 | A | A | B | A | A | A |
| Example 2-20 | 17 | B-7 | A | A | A | A | B | A |
| Example 2-21 | 19 | B-7 | A | A | A | B | — | B |
| Comparative | C-1 | B-7 | — | — | — | — | — | — |
| Example 2-1 | | | | | | | | |
| Comparative | C-2 | B-7 | — | — | — | — | — | — |
| Example 2-2 | | | | | | | | |
| Comparative | C-3 | B-7 | — | — | — | — | — | — |
| Example 2-3 | | | | | | | | |
| Comparative | C-4 | B-7 | — | — | — | — | — | — |
| Example 2-4 | | | | | | | | |
| Comparative | C-5 | B-7 | — | — | — | — | — | — |
| Example 2-5 | | | | | | | | |

| | | | | | Electric | Electric |
|---|---|---|---|---|---|---|
| | | | | | field | field |
| | | | | | strength | strength |
| | | | | | dependence | dependence |
| | Quantum | Quantum | Response | Response | of response | of response |
| | efficiency | efficiency | speed | speed | speed | speed |
| | (460 nm) | (600 nm) | (460 nm) | (600 nm) | (460 nm) | (600 nm) |
| Example 2-1 | A | A | A | A | A | A |
| Example 2-2 | A | A | A | A | A | A |
| Example 2-3 | A | A | A | A | A | A |
| Example 2-4 | A | A | A | A | A | A |
| Example 2-5 | A | A | A | A | A | A |
| Example 2-6 | A | A | A | A | A | A |
| Example 2-7 | A | A | A | A | A | A |
| Example 2-8 | A | A | A | A | A | A |
| Example 2-9 | A | A | A | A | A | A |
| Example 2-10 | A | A | A | A | A | A |
| Example 2-11 | A | A | A | A | A | A |
| Example 2-12 | A | A | A | A | A | A |
| Example 2-13 | A | A | A | A | A | A |
| Example 2-14 | A | A | A | A | A | A |
| Example 2-15 | A | A | B | B | B | B |
| Example 2-16 | A | A | A | A | A | A |
| Example 2-17 | A | A | A | A | A | A |
| Example 2-18 | C | C | C | C | B | B |
| Example 2-19 | A | A | A | A | A | A |
| Example 2-20 | B | B | A | A | B | B |
| Example 2-21 | C | C | C | C | C | B |
| Comparative | D | C | E | D | D | D |
| Example 2-1 | | | | | | |
| Comparative | E | D | E | D | D | D |
| Example 2-2 | | | | | | |
| Comparative | E | D | E | D | D | D |
| Example 2-3 | | | | | | |
| Comparative | D | C | D | C | C | C |
| Example 2-4 | | | | | | |

| | |
|---|---|
| Comparative | Not vapor-depositable |

Example 2-5

From the results shown in the above table, it was found that the desired effects were obtained in the photoelectric conversion element according to the embodiment of the present invention.

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 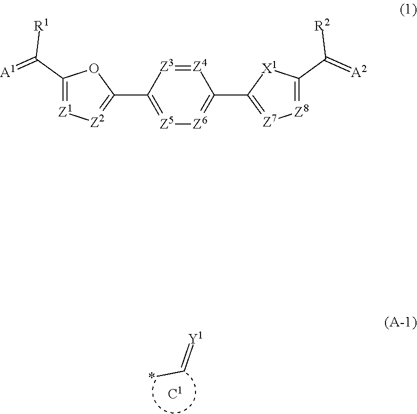
in Formula (1),
X¹ represents an oxygen atom, a sulfur atom, or a selenium atom,
Z¹ to Z⁸ each independently represent —CR^{Z1}═ or —N═,
R^{Z1} represents a hydrogen atom or a substituent,
R¹ and R² each independently represent a hydrogen atom or a substituent, and
A¹ and A² each independently represent a group represented by Formula (A-1),
in Formula (A-1),
* represents a bonding position,
C¹ represents a ring which contains two or more carbon atoms and may have a substituent,
Y¹ represents a sulfur atom, an oxygen atom, ═NR^{Y1}, or ═CR^{Y2}R^{Y3},
R^{Y1} represents a hydrogen atom or a substituent,
R^{Y2} and R^{Y3} each independently represent a cyano group, —SO₂R^{Y4}, —COOR^{Y5}, or —COR^{Y6}, and
R^{Y4}, R^{Y5}, and R^{Y6} each independently represent an aliphatic hydrocarbon group which may have a substituent, an aromatic ring group which may have a substituent, or an aliphatic heterocyclic group which may have a substituent.

2. The photoelectric conversion element according to claim 1,
wherein at least one of Z¹, . . . , or Z⁸ is represented by —CR^{Z2}═,
provided that R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃, * represents a bonding position,
R^{Si1}'s each independently represent an aliphatic hydrocarbon group which may have a substituent, or an aromatic ring group which may have a substituent, and
the aliphatic hydrocarbon group and the acyl group, represented by R^{Z2}, and the aliphatic hydrocarbon group represented by R^{Si1} may have an ether oxygen atom.

3. The photoelectric conversion element according to claim 2,
wherein R^{Z2} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from a substituent group S, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃,
provided that the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have at least one of a halogen atom or an ether oxygen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by *—Si(R^{Si2})₃,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have at least one of a halogen atom or an ether oxygen atom, and
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, represented by R^{Si2}, may have at least one of a halogen atom or an ether oxygen atom.

4. The photoelectric conversion element according to claim 3,
wherein R^{Z2} represents a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from a substituent group T, an acyl group having 2 or 3 carbon atoms, an aromatic ring group which may have a substituent selected from the substituent group T, and an aliphatic heterocyclic group which may have a substituent selected from the substituent group T,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by *—Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

5. The photoelectric conversion element according to claim 1,
wherein the group represented by Formula (A-1) is a group represented by Formula (C-1) or a group represented by Formula (C-2), 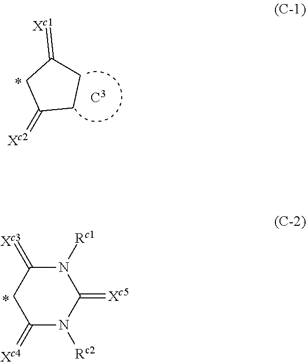
in Formula (C-1) and Formula (C-2),
* represents a bonding position,
in Formula (C-1),
C³ represents an aromatic ring which may have a substituent, and
X^{c1} and X^{c2} each independently represent an oxygen atom, a sulfur atom, ═NR^{X1}, or ═CR^{X2}R^{X3},
in Formula (C-2),
X^{c3} to X^{c5} each independently represent an oxygen atom, a sulfur atom, ═NR^{X1}, or ═CR^{X2}R^{X3},
R^{c1} and R^{c2} each independently represent a hydrogen atom or a substituent,
R^{X1} represents a hydrogen atom or a substituent,
R^{X2} and R^{X3} each independently represent a cyano group, —SO₂R^{X4}, —COOR^{X5}, or —COR^{X6}, and
R^{X4}, R^{X5}, and R^{X6} each independently represent an aliphatic hydrocarbon group which may have a substituent, an aromatic ring group which may have a substituent, or an aliphatic heterocyclic group which may have a substituent.

6. The photoelectric conversion element according to claim 5,
wherein the group represented by Formula (A-1) is the group represented by Formula (C-2).

7. The photoelectric conversion element according to claim 1,
wherein X¹ is an oxygen atom.

8. The photoelectric conversion element according to claim 1,
wherein the photoelectric conversion film further contains an n-type organic semiconductor, and
the photoelectric conversion film has a bulk heterojunction structure formed in a state in which the compound represented by Formula (1) and the n-type organic semiconductor are mixed with each other.

9. The photoelectric conversion element according to claim 8,
wherein the n-type organic semiconductor includes fullerenes selected from the group consisting of a fullerene and derivatives of the fullerene.

10. The photoelectric conversion element according to claim 1,
wherein the photoelectric conversion film further contains a coloring agent.

11. The photoelectric conversion element according to claim 1,
wherein the photoelectric conversion film further contains a p-type organic semiconductor.

12. The photoelectric conversion element according to claim 1, further comprising:
one or more interlayers between the conductive film and the transparent conductive film, in addition to the photoelectric conversion film.

13. An imaging element comprising:
the photoelectric conversion element according to claim 1.

14. An optical sensor comprising:
the photoelectric conversion element according to claim 1.

15. A compound represented by Formula (1), 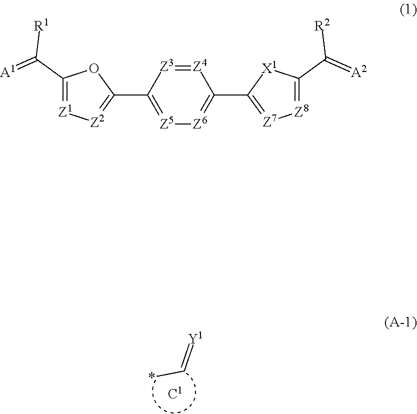
in Formula (1),
X¹ represents an oxygen atom, a sulfur atom, or a selenium atom,
Z¹ to Z⁸ each independently represent —CR^{Z1}═ or —N═,
R^{Z1} represents a hydrogen atom or a substituent,
R¹ and R² each independently represent a hydrogen atom or a substituent, and
A¹ and A² each independently represent a group represented by Formula (A-1), and
in Formula (A-1),
* represents a bonding position,
C¹ represents a ring which contains two or more carbon atoms and may have a substituent,
Y¹ represents a sulfur atom, an oxygen atom, ═NR^{Y1}, or ═CR^{Y2}R^{Y3},
R^{Y1} represents a hydrogen atom or a substituent,
R^{Y2} and R^{Y3} each independently represent a cyano group, —SO₂R^{Y4}, —COOR^{Y5}, or —COR^{Y6}, and
R^{Y4}, R^{Y5}, and R^{Y6} each independently represent an aliphatic hydrocarbon group which may have a substituent, an aromatic ring group which may have a substituent, or an aliphatic heterocyclic group which may have a substituent.

16. The compound according to claim 15,
wherein at least one of Z¹, . . . , or Z⁸ is represented by —CR^{Z2}═,
provided that R^{Z2}'s each independently represent an aliphatic hydrocarbon group which may have a substituent, an acyl group which may have a substituent, an aromatic ring group which may have a substituent, an aliphatic heterocyclic group which may have a substituent, or a group represented by *—Si(R^{Si1})₃, * represents a bonding position,
R^{Si1}'s each independently represent an aliphatic hydrocarbon group which may have a substituent, or an aromatic ring group which may have a substituent, and
the aliphatic hydrocarbon group and the acyl group, represented by R^{Z2}, and the aliphatic hydrocarbon group represented by R^{Si1} may have an ether oxygen atom.

17. The compound according to claim 16,
wherein R^{Z2} represents a linear aliphatic hydrocarbon group having 1 to 3 carbon atoms, a branched aliphatic hydrocarbon group having 3 to 5 carbon atoms, a cyclic aliphatic hydrocarbon group having 3 to 8 carbon atoms, which may have a substituent selected from a substituent group S, an acyl group having 2 to 5 carbon atoms, which may have a halogen atom, an aromatic ring group which may have a substituent selected from the substituent group S, an aliphatic heterocyclic group which may have a substituent selected from the substituent group S, or a group represented by *—Si(R^{Si2})₃,
provided that the linear aliphatic hydrocarbon group having 1 to 3 carbon atoms, the branched aliphatic hydrocarbon group having 3 to 5 carbon atoms, and the cyclic aliphatic hydrocarbon group having 3 to 8 carbon atoms may have at least one of a halogen atom or an ether oxygen atom,
the substituent group S: a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, a halogen atom, and a group represented by *—Si(R^{Si2})₃,
R^{Si2}'s each independently represent a linear aliphatic hydrocarbon group having 1 to 3 carbon atoms, a branched aliphatic hydrocarbon group having 3 or 4 carbon atoms, a cyclic aliphatic hydrocarbon group having 3 to 8 carbon atoms, which may have a substituent selected from the substituent group S, or an aromatic ring group which may have a substituent selected from the substituent group S,
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms in the substituent group S may have at least one of a halogen atom or an ether oxygen atom, and
the linear aliphatic hydrocarbon group having 1 to 3 carbon atoms, the branched aliphatic hydrocarbon group having 3 or 4 carbon atoms, and the cyclic aliphatic hydrocarbon group having 3 to 8 carbon atoms, represented by R^{Si2}, may have at least one of a halogen atom or an ether oxygen atom.

18. The compound according to claim 17,
wherein R^{Z2} represents a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from a substituent group T, an acyl group having 2 or 3 carbon atoms, an aromatic ring group which may have a substituent selected from the substituent group T, and an aliphatic heterocyclic group which may have a substituent selected from the substituent group T,
the substituent group T: a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, a halogen atom, and a group represented by *—Si(R^{Si3})₃,
R^{Si3}'s each independently represent a linear aliphatic hydrocarbon group having 1 or 2 carbon atoms, a branched aliphatic hydrocarbon group having 3 or 4 carbon atoms, a cyclic aliphatic hydrocarbon group having 3 to 6 carbon atoms, which may have a substituent selected from the substituent group T, or an aromatic ring group which may have a substituent selected from the substituent group T.

19. The compound according to claim 15,
wherein the group represented by Formula (A-1) is a group represented by Formula (C-1) or a group represented by Formula (C-2), 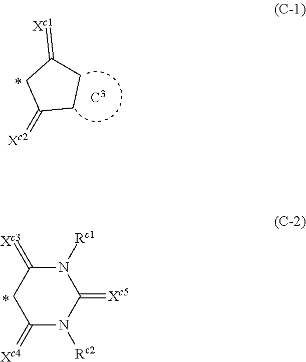
in Formula (C-1) and Formula (C-2),
* represents a bonding position,
in Formula (C-1),
C³ represents an aromatic ring which may have a substituent, and
X^{c1} and X^{c2} each independently represent an oxygen atom, a sulfur atom, ═NR^{X1}, or ═CR^{X2}R^{X3},
in Formula (C-2),
X^{c3} to X^{c5} each independently represent an oxygen atom, a sulfur atom, ═NR^{X1}, or ═CR^{X2}R^{X3},
R^{c1} and R^{c2} each independently represent a hydrogen atom or a substituent,
R^{X1} represents a hydrogen atom or a substituent,
R^{X2} and R^{X3} each independently represent a cyano group, —SO₂R^{X4}, —COOR^{X5}, or —COR^{X6}, and
R^{X4}, R^{X5}, and R^{X6} each independently represent an aliphatic hydrocarbon group which may have a substituent, an aromatic ring group which may have a substituent, or an aliphatic heterocyclic group which may have a substituent.

20. The compound according to claim 19,
wherein the group represented by Formula (A-1) is the group represented by Formula (C-2).

21. The compound according to claim 15,
wherein X¹ is an oxygen atom.
